(19) United States
(12) Patent Application Publication
(10) Pub. No.: US 2024/0398085 A1
(43) Pub. Date: Dec. 5, 2024

(54) HAIR CARE DEVICE

(71) Applicant: Panasonic Intellectual Property Management Co., Ltd., Osaka (JP)

(72) Inventors: Aya ISHIHARA, Osaka (JP); Miei KINOSHITA, Shiga (JP); Hiroyuki INOUE, Shiga (JP); Yuki CHIKAZAWA, Nara (JP)

(21) Appl. No.: 18/259,712

(22) PCT Filed: Dec. 24, 2021
(86) PCT No.: PCT/JP2021/048152

§ 371 Date: Jul. 19, 2024

(30) Foreign Application Priority Data
Feb. 5, 2021 (JP) 2021-017489

(51) Int. Cl. A45D 20/12; A45D 44/00

(52) U.S. Cl. CPC A45D 20/12; A45D 44/00; A45D 2044/007; A45D 2200/202

(57) ABSTRACT

Hair dryer (1) as a hair care device includes heat application unit (30) that applies heat to hair of a user, component generation unit (40) that generates a component that acts on the hair, and controller (80) that controls operations of heat application unit (30) and component generation unit (40). Component generation unit (40) is at least one of an ion generation unit that generates ions, an acidic component generation unit that generates an acidic component, and a charged fine particle water generation unit that generates charged fine particle water. Controller (80) controls a component amount of the component generated by component generation unit (40) based on hair characteristics of the user.

### TECHNICAL FIELD

The present disclosure relates to a hair care device.

### BACKGROUND ART

Conventionally, there is a hair care device such as a hair dryer that not only merely dries a user's hair but also applies an effective component for the user's hair. For example, PTL 1 discloses a technique related to a hair dryer in which ions are applied to hair as an effective component, and an amount of the component is adjusted based on usage time in addition to settings by the user.

### CITATION LIST

### Patent Literature

PTL 1: Unexamined Japanese Patent Publication No. 2019-58484

### SUMMARY OF THE INVENTION

The degrees of waviness and a curl of hair (curly hair) vary depending on the user.

Therefore, even w % ben amount of ions as an effective component for hair is adjusted as in the hair dryer disclosed in PTL 1, it may not act effectively depending on the degree of the waviness or the like of the user's hair. In other words, the hair dryer disclosed in PTL 1 does not necessarily achieve a finish of hair desired by the user suitable for hair characteristics such as the waviness of the hair. Further, in the hair dryer disclosed in PTL 1, only ions are shown as an effective component for hair.

The present disclosure provides a hair care device that easily provides a finish of hair desired by a user.

A hair care device according to one aspect of the present disclosure includes a heat application unit that applies heat to a user's hair, a component generation unit that generates a component acting on the hair, and a controller that controls operations of the heat application unit and the component generation unit. The component generation unit is at least one of an ion generation unit that generates ions, an acidic component generation unit that generates an acidic component, and a charged fine particle water generation unit that generates charged fine particle water. The controller controls a component amount of the component generated by the component generation unit based on the hair characteristics of the user.

According to the present disclosure, it is possible to provide a hair care device that easily provides a finish of hair desired by a user.

### BRIEF DESCRIPTION OF THE DRAWINGS

FIG. 1 is a schematic perspective view illustrating a configuration of a hair dryer according to a first exemplary embodiment.

FIG. 2 is a schematic sectional view illustrating the configuration of the hair dryer according to the first exemplary embodiment.

FIG. 3A is a diagram illustrating a configuration of a first electrostatic atomization device as an example of a component generation device that can be adopted as a component generation unit.

FIG. 3B is a diagram illustrating a configuration of a second electrostatic atomization device as an example of a component generation device that can be adopted as a component generation unit.

FIG. 3C is a diagram illustrating a configuration of a third electrostatic atomization device as an example of a component generation device that can be adopted as a component generation unit.

FIG. 4A is a view related to a first installation position example of a wetting detection sensor and an illumination unit.

FIG. 4B is a view related to a second installation position example of the wetting detection sensor and the illumination unit.

FIG. 4C is a view related to a third installation position example of the wetting detection sensor and the illumination unit.

FIG. 5 is a block diagram illustrating a configuration of a controller of the hair dryer according to the first exemplary embodiment.

FIG. 6A is a schematic view illustrating hair in a case where a hair characteristic is a curl of hair.

FIG. 6B is a schematic view illustrating hair in a case where the hair characteristic is the curl of hair.

FIG. 6C is a schematic view illustrating hair in a case where the hair characteristic is the curl of hair.

FIG. 6D is a schematic view illustrating hair in a case where the hair characteristic is the curl of hair.

FIG. 7A is a schematic view illustrating hair in a case where the hair characteristic is waviness of hair.

FIG. 7B is a schematic view illustrating hair in a case where the hair characteristic is the waviness of hair.

FIG. 8A is a schematic view illustrating a level of the curl or waviness of hair.

FIG. 8B is a schematic view illustrating the level of the curl or waviness of hair.

FIG. 8C is a schematic view illustrating the level of the curl or waviness of hair.

FIG. 8D is a schematic view illustrating the level of the curl or waviness of hair.

FIG. 9A is a table illustrating an amount of a component set for each waviness of hair and for each hair condition.

FIG. 9B is a table illustrating the amount of a component set for each waviness of hair and for each hair condition.

FIG. 10A is a view illustrating a first example of a component output ratio set for each curl of hair relative to a type of a component applied to hair.

FIG. 10B is a graph illustrating the first example of the component output ratio set for each curl of hair relative to the type of the component applied to the hair.

FIG. 11A is a view illustrating a second example of the component output ratio set for each curl of hair relative to the type of a component applied to the hair.

FIG. 11B is a graph illustrating the second example of the component output ratio set for each curl of hair relative to the type of the component applied to the hair.

FIG. 12A is a table illustrating the amount of a component set for each part of hair and for each hair characteristic.

FIG. 12B is a table illustrating the amount of a component set for each part of hair and for each hair characteristic.

FIG. 13 is a timing chart illustrating an example of a relationship between a component amount of charged fine particle water and curl detection.

FIG. 14 is a timing chart illustrating an example of a relationship between an amount of components such as zinc fine particles and curl detection.

FIG. 15 is a timing chart illustrating an example of a relationship between an amount of components such as moisture and curl detection.

FIG. 16 is a timing chart illustrating an example of a relationship between an amount of components such as a moisturizing component and curl detection.

FIG. 17A is a view illustrating a first input screen of a first example of an input screen.

FIG. 17B is a view illustrating a second input screen of the first example of the input screen.

FIG. 17C is a view illustrating a third input screen of the first example of the input screen.

FIG. 18A is a view illustrating a first example of an output screen at the time when the middle of hair is dried.

FIG. 18B is a view illustrating a first example of the output screen at the time when the root of hair is dried.

FIG. 19 is a view illustrating a second example of the output screen.

FIG. 20A is a view illustrating a third example of the output screen.

FIG. 20B is a view illustrating the third example of the output screen.

FIG. 21 is a view illustrating a fourth example of the output screen.

FIG. 22A is a view illustrating a first input screen of a second example of the input screen.

FIG. 22B is a view illustrating a second input screen of the second example of the input screen.

FIG. 23 is a table illustrating a setting example in a case where the component amount is changed for each part of hair.

FIG. 24 is a flowchart illustrating a step of determining an end of drying based on the waviness or the like.

FIG. 25 is a table illustrating a principle for determining whether or not hair is wet.

FIG. 26 is a table illustrating criteria for determining whether or not hair is wet.

FIG. 27 is a timing chart illustrating an example of a relationship between an application amount of charged fine particles and a degree of dryness.

FIG. 28 is a timing chart illustrating an example of a relationship between an application amount of a cosmetic and the degree of dryness.

FIG. 29 is a timing chart illustrating an example of a relationship between application amounts of two kinds of cosmetics and the degree of dryness.

FIG. 30 is a timing chart illustrating an example of a relationship between an air volume and the degree of dryness.

FIG. 31 is a schematic perspective view illustrating a configuration of a first example of a hair dryer according to a second exemplary embodiment.

FIG. 32 is a schematic perspective view illustrating a configuration of a second example of the hair dryer according to the second exemplary embodiment.

FIG. 33A is a flowchart illustrating a control step in the second exemplary embodiment.

FIG. 33B is a flowchart illustrating the control step in the second exemplary embodiment.

FIG. 34 is a graph illustrating an effect of the control step in the second exemplary embodiment.

FIG. 35A is a view illustrating a fifth example of the output screen.

FIG. 35B is a view illustrating the fifth example of the output screen.

FIG. 36 is a schematic sectional view illustrating a configuration of a hair dryer according to a third exemplary embodiment.

### DESCRIPTION OF EMBODIMENT

Hereinafter, a hair care device according to an exemplary embodiment of the present disclosure will be described in detail with reference to the drawings. However, unnecessary detailed description may be omitted. For example, detailed descriptions of already well-known matters or redundant descriptions of substantially the same configuration may be omitted. Note that, the accompanying drawings and the following description are merely presented to help those skilled in the art fully understand the present disclosure, and are not intended to limit the subject matters described in the scope of claims.

### FIRST EXEMPLARY EMBODIMENT

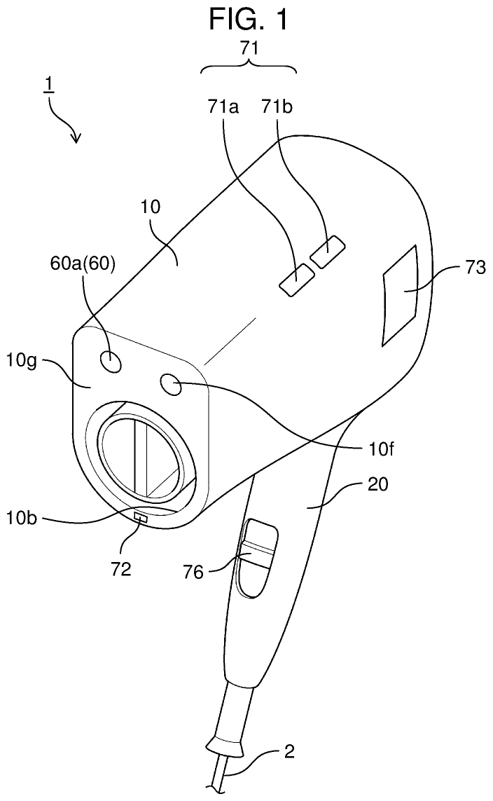

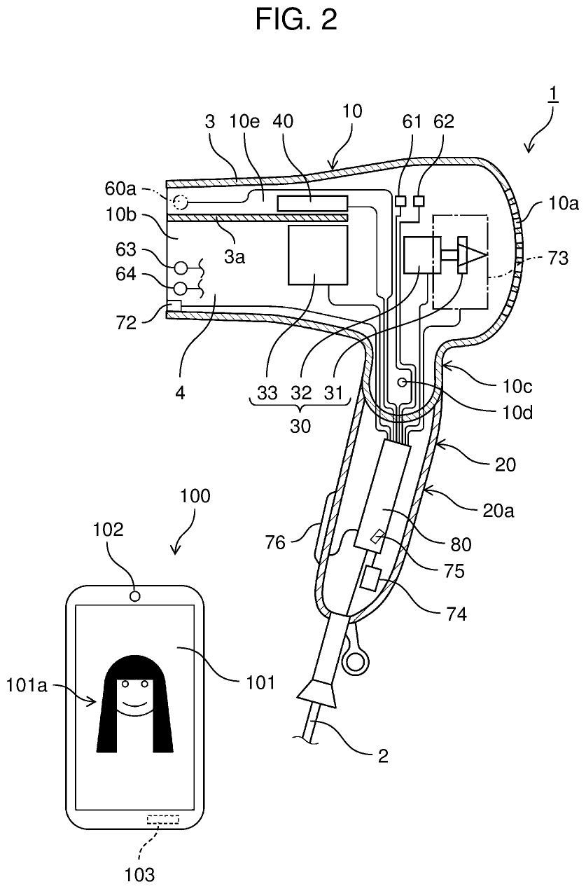

FIG. 1 is a schematic perspective view illustrating a configuration of hair dryer 1 as a hair care device according to the first exemplary embodiment. Hair dryer 1 includes main body 10 that sends warm air toward a user, and grip part 20 as a portion gripped by the user's hand during use. FIG. 2 is a schematic sectional view illustrating the configuration of hair dryer 1 cut along an air blowing direction so as to include main body 10 and grip part 20.

Main body 10 includes housing 3 forming an outer wall in which a plurality of divided bodies are joined together. Inside housing 3, air blowing flow channel 4 is formed from suction port 10a provided at one end portion in a longitudinal direction to discharge port 10b provided at the other end portion. As shown in FIG. 2, main body 10 and grip part 20 are pivotably connected by connecting part 10c with respect to connecting shaft 10d.

For example, when hair dryer 1 is not in use, grip part 20 is folded against main body 10 so as to be substantially parallel to an axial direction of main body 10 extending in the air blowing direction. In grip part 20, power supply cord 2 is pulled out from an end portion opposite to connecting part 10c.

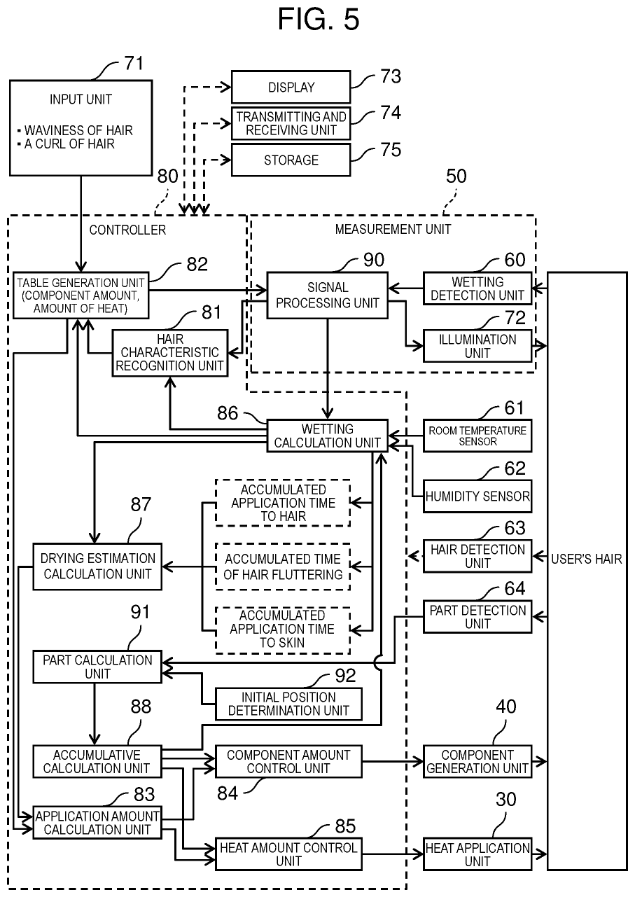

To start with, hair dryer 1 includes heat application unit 30, component generation unit 40, measurement unit 50 (see FIG. 5), input unit 71, and display 73.

Heat application unit 30 applies heat to the user's hair. In the present exemplary embodiment, heat application unit 30 is an air blower that generates warm air to be sent to the user's hair. Heat application unit 30 includes, for example, fan 31, motor 32, and heating unit 33. Fan 31 is disposed on an upstream side in air blowing flow channel 4 and rotates when motor 32 is driven. When fan 31 rotates, an air flow is formed to flow into air blowing flow channel 4 from outside via suction port 10a, pass through air blowing flow channel 4, and is discharged from discharge port 10b to outside. Heating unit 33 is disposed downstream of fan 31 and heats the air flow sent from fan 31. When heating unit 33 is operated, the air flow formed by fan 31 is heated, and warm air is blown out from discharge port 10b. Heating unit 33 may be, for example, a heater in which an electric resistor having a strip shape and a corrugated plate shape is wound along an inner periphery of housing 3.

Component generation unit 40 generates a component that acts on the user's hair. Here, the component that acts on hair refers to a so-called beauty component that can effectively act on at least hair quality of the user. Examples of the component include, for example, an agent or an organic substance, negative ions, metal fine particles, and charged fine particle water. The agent or organic substance is, for example, a moisturizing component (a moisturizing agent), a repairing component (a repairing agent), a coating component (a coating agent), or a treatment component (a treatment agent). The moisturizing component is, for example, 1.3-butylene glycol, glycerin, panthenol, ceramide, hyaluronic acid, honey, or a polysaccharide. The repair component is, for example, hydrolyzed collagen, hydrolyzed keratin, an amino acid, hair protection protein, a polypeptide, cholesterol, a cationic surfactant or an organic acid. The coating component is, for example, silicon, squalane or an oily component. The treatment component is, for example, a cationic surfactant, an amino acid, a polypeptide, panthenol, or ceramide. Further, in the present exemplary embodiment, an acidic component as a component that effectively acts on the hair is, for example, citric acid or succinic acid. Furthermore, the charged fine particle water is an electrically charged nano-sized water particle containing OH radicals. In the present exemplary embodiment, component generation unit 40 is at least one of an ion generation unit that generates ions, an acidic component generation unit that generates an acidic component, and a charged fine particle water generation unit that generates charged fine particle water.

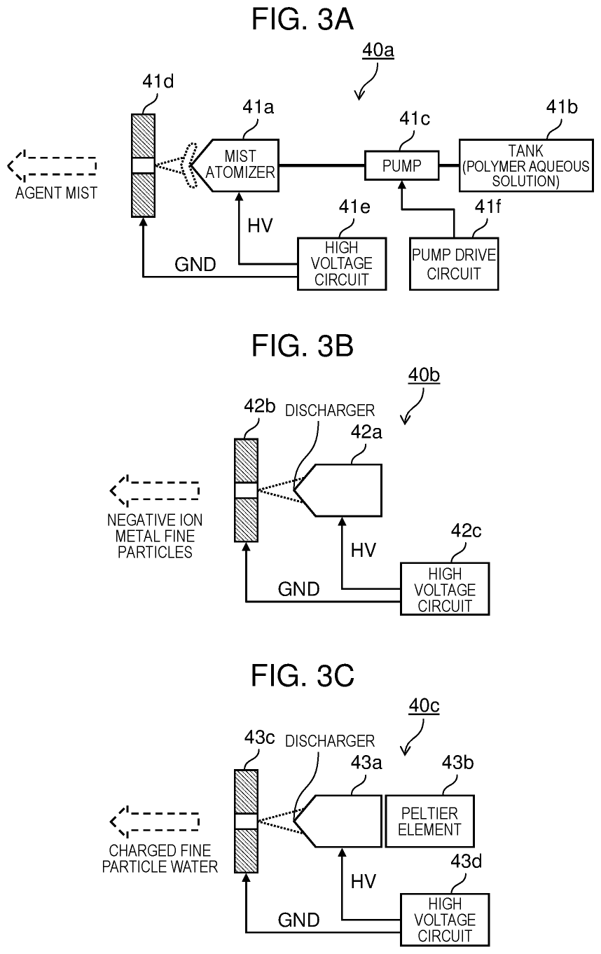

FIGS. 3A to 3C are schematic diagrams illustrating configurations of various component generation devices that can be adopted as component generation unit 40. FIG. 3A is a diagram illustrating a configuration of first electrostatic atomization device 40a as an example of an agent spraying device that uses an agent or an organic substance as a component acting on hair. First electrostatic atomization device 40a includes mist atomizer 41a, tank 41b, pump 41c, GND electrode 41d, high-voltage circuit 41e, and pump drive circuit 41f. Mist atomizer 41a is a discharger formed to hold a liquid as an agent or an organic substance. Tank 41b stores an aqueous solution containing, for example, a polymer as an agent or an organic substance. Pump 41c is installed in a pipe connecting tank 41b and mist atomizer 41a, and sends a polymer aqueous solution stored in tank 41b to mist atomizer 41a. High-voltage circuit 41e applies a high voltage (HV) to mist atomizer 41a.

Pump drive circuit 41f controls driving of pump 41c. High-voltage circuit 41e and pump drive circuit 41f are controlled by component amount control unit 84 (see FIG. 5) in controller 80 to be described in detail below. When a high voltage is applied between mist atomizer 41a and GND electrode 41d, corona discharge or the like occurs, and an agent mist containing a polymer is generated by this discharge action. Note that, the agent spraying device that uses an agent or an organic substance as the component acting on hair is not limited to an electrostatic atomization device such as first electrostatic atomization device 40a, and may be an ultrasonic atomization device, a centrifugal pump, or the like.

FIG. 3B is a diagram illustrating a configuration of second electrostatic atomization device 40b as an example of a component generation device that use negative ions or metal fine particles as a component acting on hair. Second electrostatic atomization device 40b includes discharger 42a. GND electrode 42b, and high-voltage circuit 42c. High-voltage circuit 42c is controlled by component amount control unit 84 in the same manner as the configuration of first electrostatic atomization device 40a. When a high voltage is applied between discharger 42a and GND electrode 41d, for example, corona discharge or the like occurs, and negative ions negatively charged based on moisture in the air are generated by this discharge action. In this case, second electrostatic atomization device 40b is the ion generation unit in the present exemplary embodiment.

FIG. 3C is a diagram illustrating a configuration of third electrostatic atomization device 40c as an example of a component generation device that uses charged fine particle water as a component acting on hair. Third electrostatic atomization device 40c includes discharger 43a, Peltier element 43b as a dew condensation unit, GND electrode 43c, and high-voltage circuit 43d. High-voltage circuit 43d is controlled by component amount control unit 84 in the same manner as the configuration of first electrostatic atomization device 40a. When a high voltage is applied between discharger 43a and GND electrode 43c, corona discharge or the like occurs, and charged fine particle water based on moisture in the air are generated by this discharge action. In this case, third electrostatic atomization device 40c is the charged fine particle water generation unit in the present exemplary embodiment.

For example, when component generation unit 40 in the present exemplary embodiment is third electrostatic atomization device 40c, as shown in FIG. 2, partition plate 3a that forms branch channel 10e extending in parallel with air blowing flow channel 4 is installed inside housing 3 of main body 10. Air blowing flow channel 4 allows an air flow passing through heating unit 33 to flow, whereas branch channel 10e allows an air flow not passing through heating unit 33 to flow. Then, third electrostatic atomization device 40c is installed in branch channel 10e. Further, front surface portion 10g that is a part of main body 10 and faces hair H during, for example, a drying operation, has component discharge port 10f. Component discharge port 10f communicates with branch channel 10e, and discharges the component generated by component generation unit 40 to outside. Note that, component generation unit 40 may be at least one of first electrostatic atomization device 40a, second electrostatic atomization device 40b, and third electrostatic atomization device 40c. In other words, a plurality of component generation units 40 may be provided for each component to be applied.

Measurement unit 50 (see FIG. 5) measures or photographs the user's hair, and transmits signal-processed information to controller 80. In the present exemplary embodiment, measurement unit 50 adopts a configuration that measures the user's hair. In this case, measurement unit 50 includes wetting detection unit 60, illumination unit 72, and signal processing unit 90 (see FIG. 5).

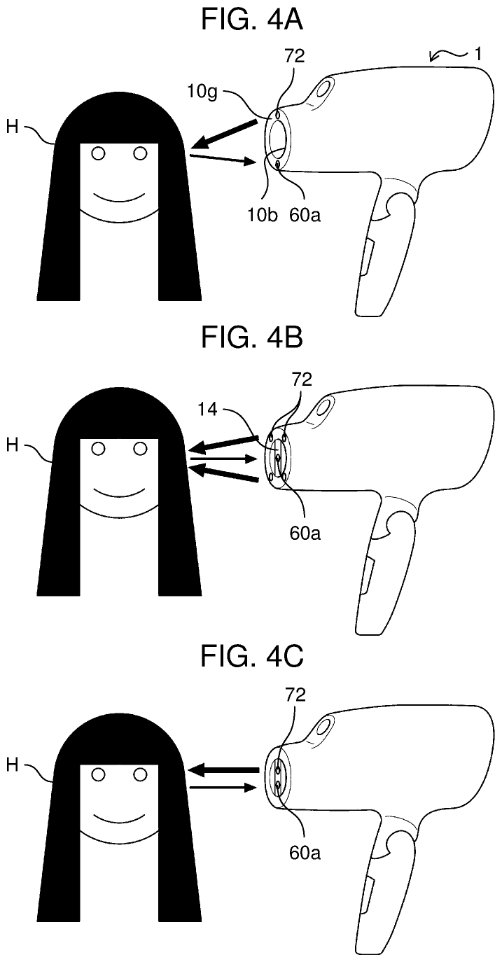

Wetting detection unit 60 detects the parameters that can be referred to for obtaining information on wetting of the user's hair. In the present exemplary embodiment, wetting detection unit 60 is wetting detection sensor 60a having at least an absorption wavelength (for example, 1450 nm) of water as a hair measurement value. Specifically, wetting detection sensor 60a may be a photodiode. Illumination unit 72 is a component paired with wetting detection sensor 60a that is, for example, a photodiode, and emits light having at least an absorption wavelength of water. Note that, signal processing unit 90 will be described in conjunction with the following matters related to controller 80. FIGS. 4A to 4C are schematic diagrams for explaining a relationship between each installation position of wetting detection sensor 60a and illumination unit 72. Each installation position of wetting detection sensor 60a and illumination unit 72 is an example in the case shown in FIGS. 1 and 2, and specifically, a plurality of examples as shown in FIGS. 4A to 4C are conceivable. While illumination unit 72 is a light irradiation unit, wetting detection sensor 60a is a light receiving unit that receives light emitted from illumination unit 72 and then reflected by the hair H of the user. Wetting detection sensor 60a and illumination unit 72 are installed on front surface portion 10g or nozzle part 14 (see FIGS. 4B and 4C) attached to discharge port 10b.

FIG. 4A is a diagram related to a first installation position example of wetting detection sensor 60a and illumination unit 72. In the first installation example, there is one wetting detection sensor 60a and one illumination unit 72. Wetting detection sensor 60a is installed on a part of front surface portion 10g. Illumination unit 72 is installed on a part of front surface portion 10g opposite to wetting detection sensor 60a across discharge port 10b. In this case, since wetting detection sensor 60a and illumination unit 72 are separated from each other in a distance larger than or equal to an opening diameter of discharge port 10b, an incident angle and a reflection angle of light are large.

FIG. 4B is a diagram related to a second installation position example of wetting detection sensor 60a and illumination unit 72. In the second installation example, there is one wetting detection sensor 60a, but there are a plurality of illumination units 72. Wetting detection sensor 60a is installed on nozzle part 14 so as to be positioned approximately at a center of discharge port 10b. There are, for example, four illumination units 72, installed at equal intervals from each other on front surface portion 10g. In this case, since wetting detection sensor 60a and illumination unit 72 are separated from each other to a certain extent, it is possible to increase an irradiation amount of light while ensuring the incident angle and the reflection angle of light at a certain size.

FIG. 4C is a diagram related to a third installation position example of wetting detection sensor 60a and illumination unit 72. In the third installation example, wetting detection sensor 60a and illumination unit 72 are installed one by one on nozzle part 14. In this case, since wetting detection sensor 60a and illumination unit 72 are relatively close to each other, the incident angle and the reflection angle of light are small.

Hereinafter, in the present exemplary embodiment, as an example, a description will be given on an assumption that wetting detection sensor 60a and illumination unit 72 are installed based on the second installation example shown in FIG. 4B.

Input unit 71 is, for example, a button for the user to input information on characteristics of the user's hair (hereinafter, referred to as “hair characteristics”). Here, the hair characteristics refer to waviness of the user's hair or a curl of the hair (curly hair). In the example shown in FIG. 1, input unit 71 is two input buttons, that is, hair waviness input unit 71a and hair curl input unit 71b, both installed in housing 3. Note that, input unit 71 may also include a button for simply switching an air volume, an air temperature, and the like according to the user's preference.

Display 73 is, for example, a touch panel type display screen installed in housing 3, and functions as an input screen on which a user inputs information or an output screen that displays information to the user. Note that, a state of functioning as an input screen or an output screen will be described in detail below. Further, in a case where display 73 functions as an input screen, display 73 substitutes the function performed by input unit 71, so that input unit 71 may be unnecessary in some cases.

Further, as shown in FIG. 2, hair dryer 1 includes room temperature sensor 61, humidity sensor 62, hair detection unit 63, and part detection unit 64.

Room temperature sensor 61 is a sensor for measuring a temperature in a room where hair dryer 1 is used. Room temperature sensor 61 is installed inside housing 3. An output signal from room temperature sensor 61 is transmitted to controller 80.

Humidity sensor 62 is a sensor for measuring humidity in a room where hair dryer 1 is used. Room temperature sensor 61 is installed inside housing 3. An output signal from humidity sensor 62 is transmitted to controller 80.

Hair detection unit 63 detects whether the user has hair. Hair detection unit 63 is, for example, a laser range finder or a time of flight (ToF) camera, and is installed in a part of front surface portion 10g. An output signal from hair detection unit 63 is transmitted to controller 80.

Part detection unit 64 detects a part where heat is applied to the user's hair or a part where the component described above is applied to the user's hair. Part detection unit 64 may be an orientation detection unit (an orientation sensor) with at least one axis that detects a position or an orientation of hair dryer 1, or a distance measurement unit (a distance sensor) that measures a distance to the use's hair or skin (face). Here, when part detection unit 64 is a distance measurement unit, it is installed on a part of front surface portion 10g. On the other hand, when part detection unit 64 is an orientation detection unit, it is not limited to be installed on front surface portion 10g, and may be installed inside housing 3. An output signal from part detection unit 64 is transmitted to controller 80.

FIG. 5 is a block diagram showing a configuration of controller 80 of hair dryer 1. Controller 80 controls overall operations of hair dryer 1, and at least controls operations of heat application unit 30 and component generation unit 40 based on the hair measurement value obtained from measurement unit 50. Controller 80 is installed, for example, inside housing 20a of grip part 20. Note that, controller 80 includes a computer system including a processor and a memory. Then, when the processor executes programs stored in the memory, the computer system functions as controller 80. Here, the programs executed by the processor is recorded in advance in the memory of the computer system, but may be provided by being recorded in a non-transitory recording medium such as a memory card, or may be provided through a telecommunication line such as the Internet.

To start with, controller 80 includes hair characteristic recognition unit 81, table generation unit 82, application amount calculation unit 83, component amount control unit 84, and heat amount control unit 85. Hair characteristic recognition unit 81, table generation unit 82, application amount calculation unit 83, component amount control unit 84, and heat amount control unit 85 are a block group for determining a component application amount and a heat application amount based on the hair characteristics of the user.

Hair characteristic recognition unit 81 classifies the hair characteristics of the user based on the hair measurement value obtained from measurement unit 50.

Table generation unit 82 sets the component amount of the component generated by component generation unit 40 and the amount of heat applied from heat application unit 30, and manages these set values as a table. In table generation unit 82, the component amount and the amount of heat are set for each hair characteristic classified by hair characteristic recognition unit 81.

Based on the component amount or the amount of heat set by table generation unit 82, application amount calculation unit 83 calculates the component application amount applied to hair by component generation unit 40 or the heat application amount applied to hair by heat application unit 30. In the present exemplary embodiment, application amount calculation unit 83 can execute the following two types of calculations. First, application amount calculation unit 83 calculates the component application amount for each user based on the hair characteristics of the whole hair classified by hair characteristic recognition unit 81 and the component amount set by table generation unit 82. Second, application amount calculation unit 83 calculates the component application amount or the heat application amount for each part of the hair based on the hair characteristics for each part of the hair classified by hair characteristic recognition unit 81 and the component amount set by table generation unit 82.

Component amount control unit 84 controls the operation of component generation unit 40, that is, the component amount of the component generated by component generation unit 40, based on the component application amount transmitted from application amount calculation unit 83.

Heat amount control unit 85 controls the operation of heat application unit 30, that is, the amount of heat applied from heat application unit 30 based on the heat application amount transmitted from application amount calculation unit 83.

Further, controller 80 includes wetting calculation unit 86 and drying estimation calculation unit 87. Wetting calculation unit 86 and drying estimation calculation unit 87 are a block group for reflecting a dry state of the user's hair in the component application amount and the heat application amount.

Wetting calculation unit 86 calculates wetting information about wetting of the user's hair based on the hair measurement value obtained from measurement unit 50. Here, for example, in a case where wetting detection unit 60 in measurement unit 50 is wetting detection sensor 60a, the wetting information is absorbance calculated based on signal intensity from wetting detection sensor 60a.

Drying estimation calculation unit 87 estimates the degree of dryness of the user's hair based on the wetting information calculated by wetting calculation unit 86. When the wetting information is the absorbance, drying estimation calculation unit 87 estimates the degree of dryness based on a change in absorbance. In drying estimation calculation unit 87, for example, an accumulated time (time subtraction) in which a component or heat is applied to the hair, an accumulated time (time addition) in which the hair is in a fluttering state, an accumulated time (time addition) in which a component or heat is applied to the skin (face), or the like is appropriately referred to according to the change in absorbance. The degree of dryness estimated by drying estimation calculation unit 87 is reflected in the component application amount by component amount control unit 84 or the heat application amount by heat amount control unit 85 via application amount calculation unit 83. In other words, the component application amount or the heat application amount is corrected for each degree of dryness reflecting various accumulated times.

Further, controller 80 includes part calculation unit 91, initial position determination unit 92, and accumulative calculation unit 88. Part calculation unit 91, initial position determination unit 92, and accumulative calculation unit 88 are a block group for specifying a part of the user's hair to which the component and heat are applied.

Based on the information detected by part detection unit 64 and an initial position determined by initial position determination unit 92, part calculation unit 91 estimates a part of the hair or the skin to which the heat from heat application unit 30 is applied or to which the component from component generation unit 40 is applied.

Initial position determination unit 92 determines an initial position of hair dryer 1, and transmits the initial position to part calculation unit 91.

Accumulative calculation unit 88 calculates, for each part detected by part detection unit 64, an accumulative amount of heat applied by heat application unit 30 and accumulated in the hair or an accumulative component amount applied by component generation unit 40 and accumulated in the hair. In this case, heat amount control unit 85 causes heat application unit 30 to adjust the amount of heat based on the accumulative amount of heat calculated by accumulative calculation unit 88. Specifically, heat amount control unit 85 corrects the heat application amount using data related to the accumulative amount of heat calculated by accumulative calculation unit 88, and controls the operation of heat application unit 30. On the other hand, component amount control unit 84 causes component generation unit 40 to adjust the component amount based on the accumulative component amount calculated by accumulative calculation unit 88. Specifically, component amount control unit 84 corrects the component application amount using data related to the accumulative component amount calculated by accumulative calculation unit 88, and controls the operation of component generation unit 40.

Furthermore, controller 80 is electrically connected to signal processing unit 90 included in measurement unit 50. Signal processing unit 90 controls light irradiation by illumination unit 72, processes an output of wetting detection unit 60 that is wetting detection sensor 60a, and transmits the processed output to wetting calculation unit 86 as signal intensity. Further, signal processing unit 90 may transmit the output of wetting detection unit 60 to hair characteristic recognition unit 81 as signal intensity. In this case, hair characteristic recognition unit 81 can classify the hair characteristics of the user based on the signal intensity transmitted from signal processing unit 90.

Further, as shown in FIG. 2, hair dryer 1 includes power supply switch 76. Power supply switch 76 is installed, for example, in housing 20a of grip part 20. When a user operates power supply switch 76 to turn a power supply ON, power is supplied to each part of hair dryer 1 via power supply cord 2 extending from an end portion of grip 20. Further, power supply switch 76 can also operate switching between warm air and cold air by heat application unit 30, switching of the air volume, and the like.

Furthermore, hair dryer 1 may include transmitting and receiving unit 74 and storage 75.

Transmitting and receiving unit 74 transmits signals to a communication device outside hair dryer 1 or receives signals transmitted from the communication device outside hair dryer 1 in accordance with a command from controller 80. Here, the communication device outside may be, for example, portable terminal device 100 as shown in FIG. 2. Portable terminal device 100 includes terminal display unit 101, terminal photographing unit 102, and terminal communication unit 103. Terminal display unit 101 is a touch panel screen that displays image 101a. Terminal display unit 101 is an output screen that displays information to the user, and is an input screen on which the user instructs or inputs information by touching. Terminal communication unit 103 performs transmission and reception with at least transmitting and receiving unit 74 of hair dryer 1.

Storage 75 is an information storage medium that passes various data with controller 80 and stores these data. The type of the information storage medium is not particularly limited.

Next, an operation of hair dryer 1 will be described.

As a basic operation of hair dryer 1, when the user turns the power supply ON by operating power supply switch 76 while gripping grip part 20, heat application unit 30 starts to operate. Specifically, when motor 32 is driven by electric power supply and fan 31 rotates, air is taken into air blowing flow channel 4 from suction port 10a. At the same time, when heating unit 33 generates heat, the air sent from fan 31 is heated. The heated air becomes warm air and is discharged from discharge port 10b. Further, when the user appropriately operates input unit 71, hair dryer 1 causes component generation unit 40 to generate an effective component for hair and discharge it from component discharge port 10f.

Furthermore, hair dryer 1 automatically optimizes a component application amount to be applied to the hair in accordance with the hair characteristics of the user. Hereinafter, optimization of the component application amount will be specifically described.

To start with, an example of hair characteristics assumed in the present exemplary embodiment will be described.

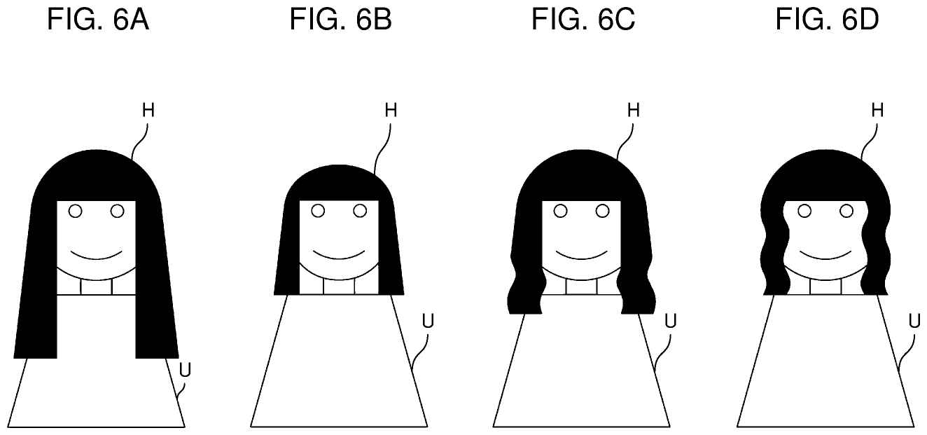

FIGS. 6A to 6D are schematic views illustrating hair H in a case where the hair characteristic of the user U is the curl of hair H. FIG. 6A shows hair H in a case where the hairstyle is long and straight and there is no curly hair. FIG. 6B shows hair H in a case where the hairstyle is short and straight and there is no curly hair. FIG. 6C shows hair H in a case where there is a curl at the tip of the hair. FIG. 6D shows hair H in a case where there is curly hair as a whole.

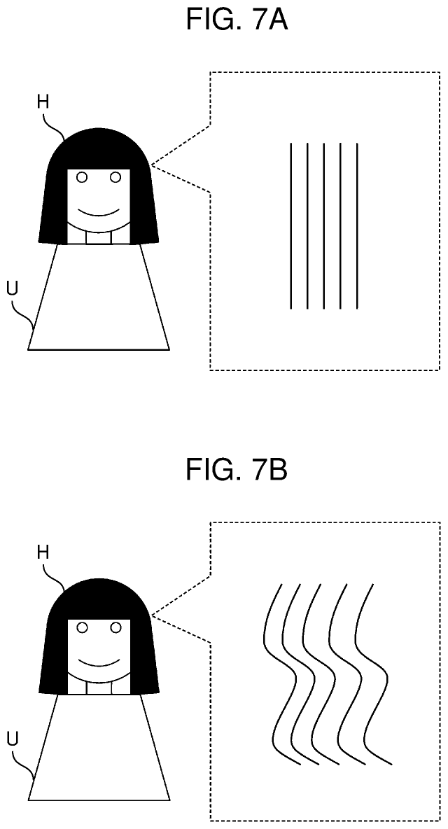

FIGS. 7A and 7B are schematic views illustrating hair H when the hair characteristic of the user U is the waviness of hair H. FIG. 7A shows hair H in a case where there is no waviness in hair H. FIG. 7B shows hair H in a case where there is waviness in hair H.

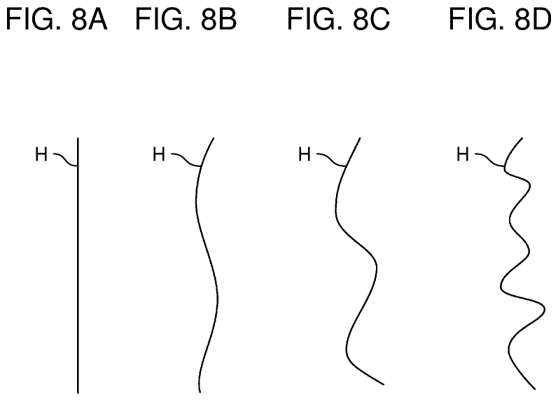

FIGS. 8A to 8D are schematic views illustrating the level of the curl or waviness of hair H. FIG. 8A shows hair H in a case of so-called straight hair without a curl or waviness in hair H. FIG. 8B shows hair H in a case where there is a curl or waviness and the level is weak. FIG. 8C shows hair H in a case where there is a curl or waviness and the level is medium. FIG. 8D shows hair H in a case where there is a curl or waviness and the level is strong.

For example, hair characteristic recognition unit 81 can classify the hair characteristics of the user U by discriminating the type and level of each hair characteristic illustrated in FIGS. 6A to 8D using wetting detection unit 60 in measurement unit 50.

Next, an example of the component amount set by table generation unit 82 for each hair characteristic classified by hair characteristic recognition unit 81 will be described.

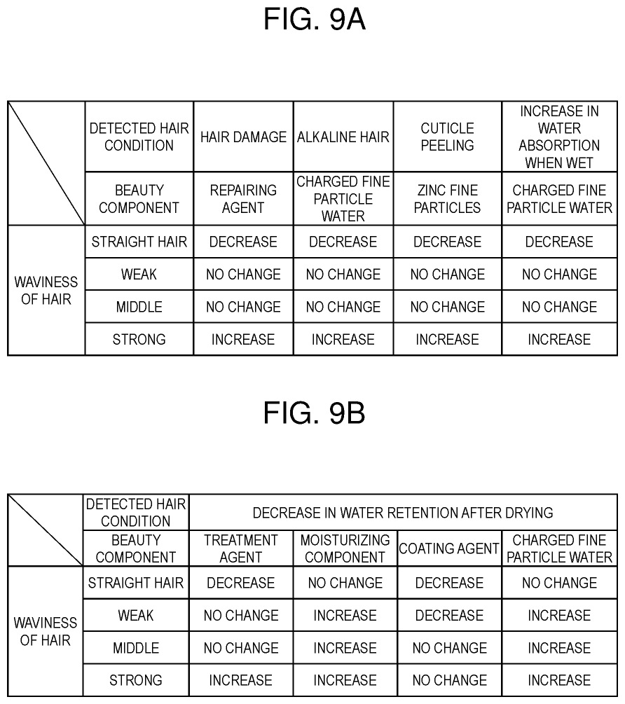

FIGS. 9A and 9B are tables illustrating examples of the amount of a component set for each waviness (curl) of the user's hair and for each hair condition. Here, the waviness of the user's hair is classified into four levels shown in FIGS. 8A to 8D as an example. Further, the hair condition is hair quality as a condition detected for each hair, and specifically, is hair damage, alkaline hair, cuticle peeling, an increase in water absorption when hair is wet, and a decrease in water retention after hair is dried.

FIG. 9A is a table illustrating a case where the hair condition is hair damage, alkaline hair, cuticle peeling, or an increase in water absorption when wet. First, a case is assumed that hair dryer 1 can apply a repair agent as a component for repairing damage to the user's hair, and controller 80 determines that the hair is greatly damaged. At this time, in a case where the hair is straight hair, controller 80 controls component generation unit 40 to apply the repairing agent in an amount smaller than the default component amount. Further, in a case where the level of the waviness is weak or medium, controller 80 controls component generation unit 40 to apply the repairing agent without changing the default component amount. Furthermore, in a case where the level of the waviness is strong, controller 80 controls component generation unit 40 to apply the repairing agent in an amount larger than the default component amount.

Further, a case is assumed that hair dryer 1 can apply charged fine particle water as a component for repairing alkaline hair of the user, and controller 80 determines that the user's hair is alkaline hair. At this time, controller 80 may control component generation unit 40 for each level of the waviness of the hair in the same manner as in a case where the hair is damaged and the repairing agent is applied.

Further, a case is assumed where hair dryer 1 can apply zinc fine particles (metal fine particles) as a component for repairing cuticle peeling occurring in the user's hair, and controller 80 determines that cuticle peeling has occurred. At this time, controller 80 may control component generation unit 40 for each level of the waviness of the hair in the same manner as in a case where the hair is damaged and the repairing agent is applied.

Furthermore, a case is assumed that hair dryer 1 can apply charged fine particle water, and controller 80 determines that the amount of water absorption when the user's hair is wet has increased. At this time, controller 80 may control component generation unit 40 for each level of the waviness of the hair in the same manner as in a case where the hair is damaged and the repairing agent is applied.

FIG. 9B is a table illustrating a case where the hair condition is a decrease in water retention after hair is dried. First, a case is assumed that hair dryer 1 can apply a treatment agent as a component for compensating for a decrease in water retention, and controller 80 determines that the water retention after the hair is dried has decreased. At this time, in a case where the hair is straight hair, controller 80 controls component generation unit 40 to apply the treatment agent in an amount smaller than the default component amount. Further, in a case where the level of the waviness is weak or medium, controller 80 controls component generation unit 40 to apply the treatment agent without changing the default component amount. Furthermore, in a case where the level of the waviness is strong, controller 80 controls component generation unit 40 to apply the treatment agent in an amount larger than the default component amount.

Further, a case is assumed that hair dryer 1 can apply a moisturizing component as a component for compensating for a decrease in water retention, and controller 80 determines that the amount of water retention after the hair is dried has decreased. At this time, in a case where the hair is straight hair, controller 80 controls component generation unit 40 to apply the moisturizing component without changing the default component amount. Further, in a case where the level of the waviness is weak, medium, or strong, controller 80 controls component generation unit 40 to apply the moisturizing component in an amount larger than the default component amount.

Further, a case is assumed that hair dryer 1 can apply a coating agent as a component for compensating for a decrease in water retention, and controller 80 determines that the amount of water retention after the hair is dried has decreased. At this time, in a case where the hair is straight hair, or in a case where the level of the waviness is weak, controller 80 controls component generation unit 40 to apply the coating agent in an amount smaller than the default component amount. Further, in a case where the level of the waviness is medium or strong, controller 80 controls component generation unit 40 to apply the coating agent without changing the default component amount.

Furthermore, a case is assumed that hair dryer 1 can apply charged fine particle water as a component for compensating for a decrease in the amount of water retention, and controller 80 determines that the amount of water retention after the hair is dried has decreased. At this time, controller 80 may control component generation unit 40 for each level of the waviness of the hair in the same manner as in a case where the moisturizing component is applied.

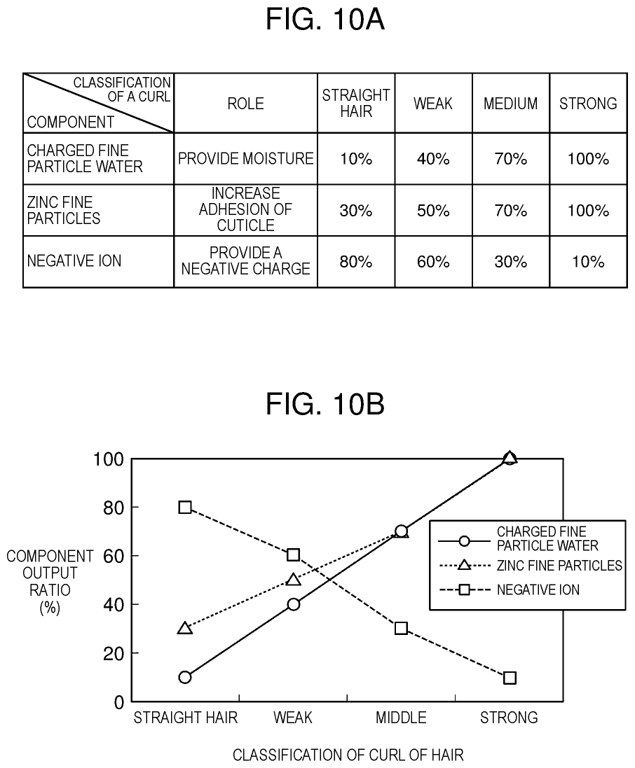

FIGS. 10A and 10B are diagrams illustrating a first example of a component output ratio set for each curl of the hair relative to the type of a component applied to the hair. FIG. 10A is a table summarizing the first example of the component output ratio (%). FIG. 10B is a graph summarizing the first example of the component output ratio (%). Since charged fine particle water can provide moisture to hair, the component amount thereof may be increased as the level of the curl becomes stronger from straight hair. Since zinc fine particles can increase adhesion of the cuticle of hair, the component amount thereof be increased as the level of the curl becomes stronger from straight hair. Since negative ions can provide a negative charge to hair, the component amount thereof may be reduced as the level of the curl becomes stronger from straight hair.

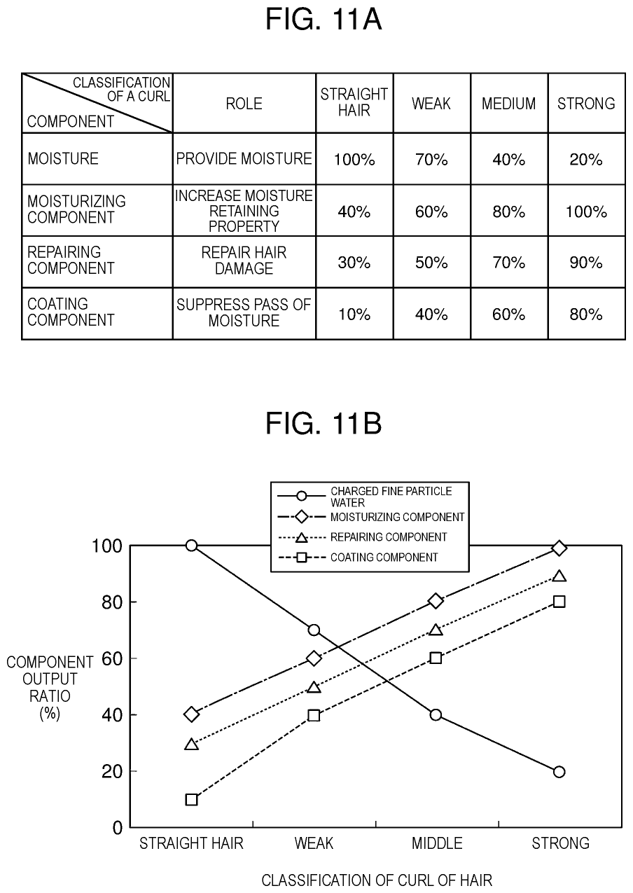

FIGS. 11A and 11B are diagrams illustrating a second example of the component output ratio set for each curl of the hair relative to the type of the component applied to the hair. FIG. 11A is a table summarizing the second example of the component output ratio (%). FIG. 11B is a graph summarizing the second example of the component output ratio (%). Since moisture can provide moisture to hair, the component amount thereof may be reduced as the level of the curl becomes stronger from straight hair. Since a moisturizing component can increase a moisture retaining property of hair, the component amount thereof may be increased as the level of the curl becomes stronger from straight hair. Since a repairing component can repair hair damage, the component amount thereof may be increased as the level of the curl becomes stronger from straight hair. Since a coating component can prevent moisture from flowing in and out of hair, the component amount thereof may be increased as the level of the curl becomes stronger from straight hair.

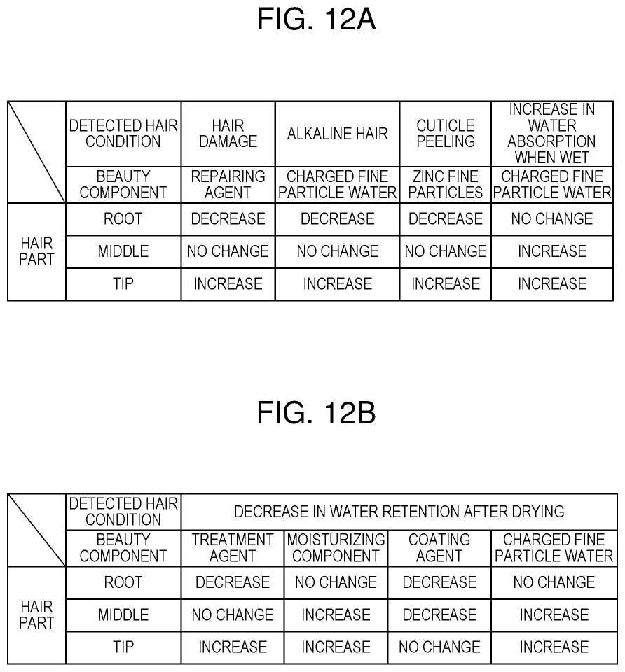

FIGS. 12A and 12B are tables illustrating examples of the amount of a component set for each part of the user's hair and for each hair characteristic. Here, the part of the user's hair shall be classified into three parts of the root, the middle, and the tip, as an example. Further, the hair condition is the same as that shown in FIGS. 9A and 9B.

FIG. 12A is a table illustrating a case where the hair condition is hair damage, alkaline hair, cuticle peeling, or an increase in water absorption when the hair is wet. First, a case is assumed that hair dryer 1 can apply a repair agent as a component for repairing damage to the user's hair, and controller 80 determines that the hair is greatly damaged. At this time, controller 80 controls component generation unit 40 to apply the repair agent to the root of the hair in an amount smaller than the default component amount. Further, controller 80 controls component generation unit 40 to apply the repair agent to the middle of the hair without changing the default component amount. Furthermore, controller 80 controls component generation unit 40 to apply the repair agent to the tip of the hair in an amount larger than the default component amount.

Further, a case is assumed that hair dryer 1 can apply charged fine particle water as a component for repairing alkaline hair of the user, and controller 80 determines that the user's hair is alkaline hair. At this time, controller 80 controls component generation unit 40 to apply the charged fine particle water to the root of the hair in an amount smaller than the default component amount. Further, controller 80 controls component generation unit 40 to apply the charged fine particle water to the middle of the hair without changing the default component amount. Furthermore, controller 80 controls component generation unit 40 to apply the charged fine particle water to the tip of the hair in an amount larger than the default component amount.

Further, a case is assumed where hair dryer 1 can apply zinc fine particles (metal fine particles) as a component for repairing cuticle peeling occurring in the user's hair, and controller 80 determines that cuticle peeling has occurred. At this time, controller 80 controls component generation unit 40 to apply the zinc fine particles to the root of the hair in an amount smaller than the default component amount. Further, controller 80 controls component generation unit 40 to apply zinc fine particles to the middle of the hair without changing the default component amount. Furthermore, controller 80 controls component generation unit 40 to apply zinc fine particles to the tip of the hair in an amount larger than the default component amount.

Furthermore, a case is assumed that hair dryer 1 can apply charged fine particle water, and controller 80 determines that the amount of water absorption when the user's hair is wet has increased. At this time, controller 80 controls component generation unit 40 to apply the charged fine particle water to the root of the hair without changing the default component amount. Further, controller 80 controls component generation unit 40 to apply the charged fine particle water to the middle and the tip of the hair in an amount larger than the default component amount.

FIG. 12B is a table illustrating a case where the hair condition is a decrease in water retention after hair is dried. First, a case is assumed that hair dryer 1 can apply a treatment agent as a component for compensating for a decrease in water retention, and controller 80 determines that the water retention after the hair is dried has decreased. At this time, controller 80 controls component generation unit 40 to apply the treatment agent to the root of the hair in an amount smaller than the default component amount. Further, controller 80 controls component generation unit 40 to apply the treatment agent to the middle of the hair without changing the default component amount. Furthermore, controller 80 controls component generation unit 40 to apply the treatment agent to the tip of the hair in an amount larger than the default component amount.

Further, a case is assumed that hair dryer 1 can apply a moisturizing component as a component for compensating for a decrease in water retention, and controller 80 determines that the amount of water retention after the hair is dried has decreased. At this time, controller 80 controls component generation unit 40 to apply the moisturizing component to the root of the hair without changing the default component amount. Further, controller 80 controls component generation unit 40 to apply the moisturizing component to the middle and the tip of the hair in an amount larger than the default component amount.

Further, a case is assumed that hair dryer 1 can apply a coating agent as a component for compensating for a decrease in water retention, and controller 80 determines that the amount of water retention after the hair is dried has decreased. At this time, controller 80 controls component generation unit 40 to apply the coating agent to the root and the middle of the hair in an amount smaller than the default component amount. Further, controller 80 controls component generation unit 40 to apply the coating agent to the tip of the hair without changing the default component amount.

Furthermore, a case is assumed that hair dryer 1 can apply charged fine particle water as a component for compensating for a decrease in the amount of water retention, and controller 80 determines that the amount of water retention after the hair is dried has decreased. At this time, controller 80 controls component generation unit 40 to apply the charged fine particle water to the root of the hair without changing the default component amount. Further, controller 80 controls component generation unit 40 to apply the charged fine particle water to the middle and the tip of the hair in an amount larger than the default component amount.

Next, a description will be given of an example of a displacement of the component amount applied by component generation unit 40 with the drying time as a time series.

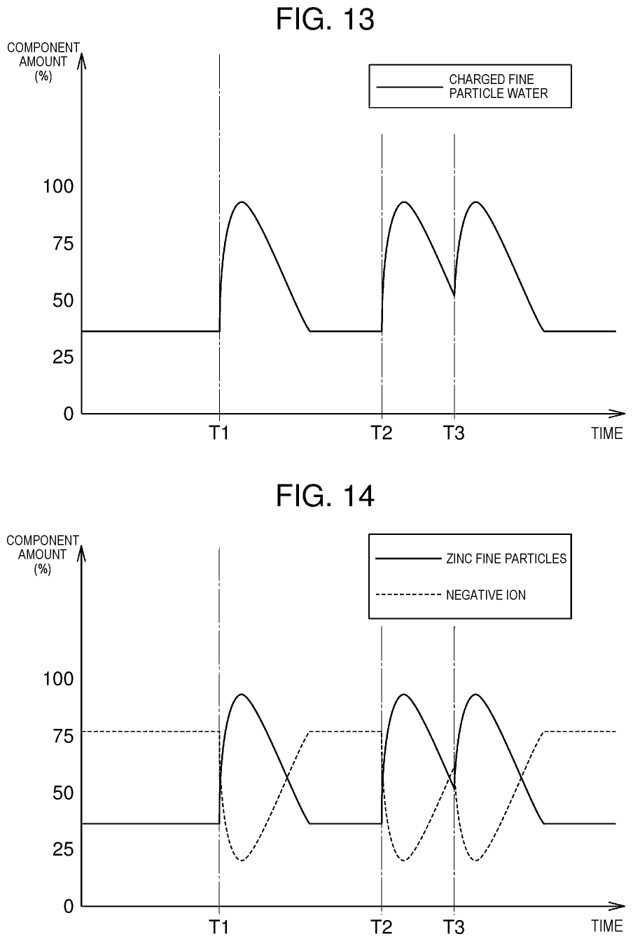

FIG. 13 is a timing chart illustrating an example of a relationship between the component amount of the charged fine particle water and a timing at which a curl in the user's hair is detected. A horizontal axis represents the drying time. The timing at which the curl is detected during the drying time is denoted as “T.” A vertical axis represents the component amount (%) of the charged fine particle water applied to the hair. During the drying operation, controller 80 determines existence or nonexistence of a curl for each part of the hair based on, for example, the output signals from part detection unit 64 and wetting detection unit 60. When detecting a curl at first timing T1, controller 80 controls component amount control unit 84 to increase the component amount from the default to a desired component amount, and then gradually decrease the component amount to return it to the default as compared with a case where the component amount is increased. The default of the component amount of the charged fine particle water may be about 35%. The component amount at the time of maximum application of the charged fine particle water may be about 90%. Thereafter, when detecting a curl at second timing T2, controller 80 causes component amount control unit 84 to execute the same control as in the case of first timing T1. In a case where the component amount is temporarily increased due to second timing 12 and third timing T3 occurs during the return to the default, controller 80 may control component amount control unit 84 to increase the component amount again before the component amount returns to the default.

FIG. 14 is a timing chart illustrating an example of a relationship between the component amounts of zinc fine particles and negative ions and the timing at which a curl in the user's hair is detected. The vertical axis, the horizontal axis, and timing T in FIG. 14 are the same as the axes and timing T in FIG. 13. First, in a case where the component is zinc fine particles, controller 80 may adjust the component amount in the same manner as in a case where the charged fine particle water shown in FIG. 13 is applied. The default of the component amount of the zinc fine particles may be about 35%. The component amount at the time of the maximum application of the zinc fine particles may be about 90%. On the other hand, in a case where the component is negative ions, controller 80 controls component amount control unit 84 to decrease the component amount from the default to a desired component amount when detecting a curl at first timing T1, and then gradually increase the component amount to return it to the default as compared with when the component amount is decreased. The default of the component amount of the negative ions may be about 75%. The component amount at the time of minimum application of negative ions may be about 20%. Thereafter, when detecting a curl at second timing T2, controller 80 causes component amount control unit 84 to execute the same control as in the case of first timing T1. In a case where the component amount is temporarily decreased due to second timing T2 and third timing T3 occurs during the return to the default, controller 80 may control component amount control unit 84 to decrease the component amount again before the component amount returns to the default.

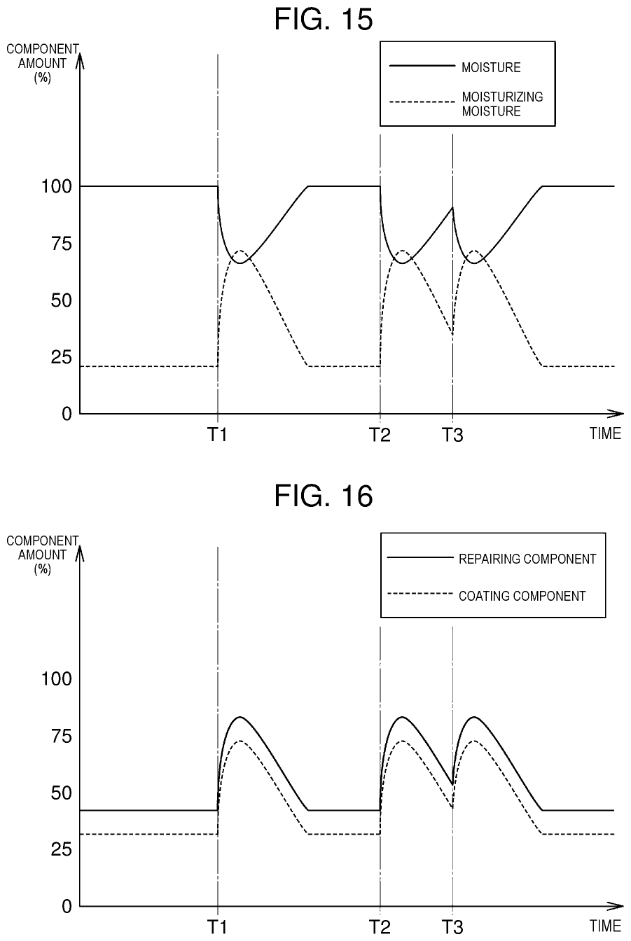

FIG. 15 is a timing chart showing an example of a relationship between the component amounts of moisture and a moisturizing component and the timing at which a curl in the user's hair is detected. The vertical axis, the horizontal axis, and timing T in FIG. 15 are the same as the axes and timing T in FIG. 13. To start with, in a case where the component is moisture, controller 80 may adjust the component amount in the same manner as in a case where the negative ions shown in FIG. 14 are applied. The default of the component amount of moisture may be 100%. The component amount at the time of the minimum application of moisture may be about 70%. On the other hand, in a case where the component is a moisturizing component, controller 80 may adjust the component amount in the same manner as in a case where the charged fine particle water shown in FIG. 13 is applied. The default of the component amount of the moisturizing component may be about 25%. The component amount at the time of the maximum application of the moisturizing component may be about 75%.

FIG. 16 is a timing chart illustrating an example of a relationship between the component amounts of a repairing component and a coating component and the timing at which a curl in the user's hair is detected. The vertical axis, the horizontal axis, and the timing T in FIG. 16 are the same as the axes and timing T in FIG. 13. In a case where the component is a repairing component and in a case where the component is a coating component, controller 80 may adjust the component amount in the same manner as in a case where the charged fine particle water shown in FIG. 13 is applied. The default of the component amount of the repairing component may be about 40%. The component amount at the time of the maximum application of the repairing component may be about 80%. On the other hand, the default of the component amount of the coating component may be about 30%. The component amount at the time of the maximum application of the coating component may be about 70%.

Next, an input screen on which a user inputs information and an output screen that displays information to the user will be described. In the present exemplary embodiment, display 73 is installed in housing 3 of main body 10. Therefore, an input screen and an output screen may be displayed on display 73. On the other hand, in a case w % here hair dryer 1 includes transmitting and receiving unit 74 that performs transmission and reception of various types of information with portable terminal device 100, an input screen and an output screen may be displayed on terminal display unit 101 of portable terminal device 100 instead of display 73. In other words, as long as hair dryer 1 includes transmitting and receiving unit 74, display 73 may not be included. In the following description, a case where an input screen and an output screen are displayed on terminal display unit 101 of portable terminal device 100 will be illustrated.

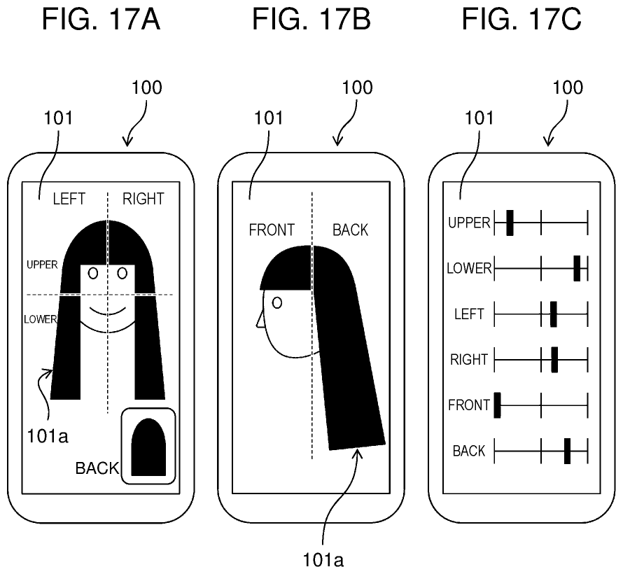

FIGS. 17A to 17C are schematic views illustrating a first example of an input screen displayed on terminal display unit 101 (or display 73). FIG. 17A shows a first input screen according to the first example. Image 101a displayed on the first input screen is a schematic view of the users front hairstyle, and is a divided image divided into upper, lower, left, and right. On the first input screen, a schematic drawing of the users rear hairstyle is also displayed. FIG. 17B shows a second input screen of the first example. Image 101a displayed on the second input screen is a schematic view of the users side hairstyle, and is a divided image divided into front and rear. FIG. 17C shows a third input screen of the first example. On the third input screen, a level adjustment screen for the user to change the setting of a certain item is displayed for each area of the divided screens displayed on the first input screen and the second input screen.

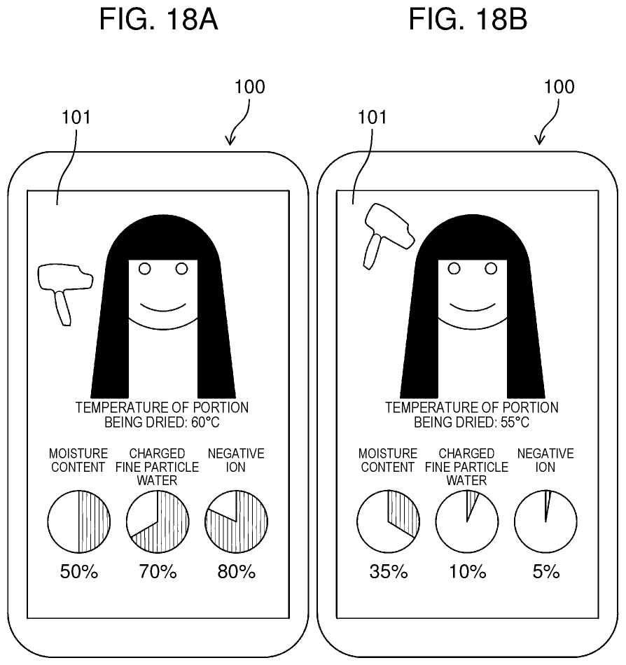

FIGS. 18A and 18B are schematic views illustrating a first example of an output screen displayed on terminal display unit 101 (or display 73). The output screen of the first example displays various states in real time related to a period in which hair dryer 1 is drying hair or a period in which hair dryer 1 is applying a component to hair. FIG. 18A is an output screen at the time when the middle of the hair is dried. FIG. 18B is an output screen at the time when the root of the hair is dried. Display items on the output screen of the first example are, for example, a part of the hair being dried, a temperature of the part being dried (a temperature of a portion being dried), the moisture content in the hair, and the amount of the component being applied, respectively, at the present time. In this example, the component amounts of charged fine particle water and negative ions are displayed as the amount of a component. As shown in FIGS. 18A and 18B, the part of the hair being dried may be visually indicated to the user by displaying a schematic drawing of a hair dryer relative to the schematic drawing of the hair. Similarly, the moisture content and the amount of a component may be visually indicated to the user by displaying a pie chart, for example, in addition to numerical values.

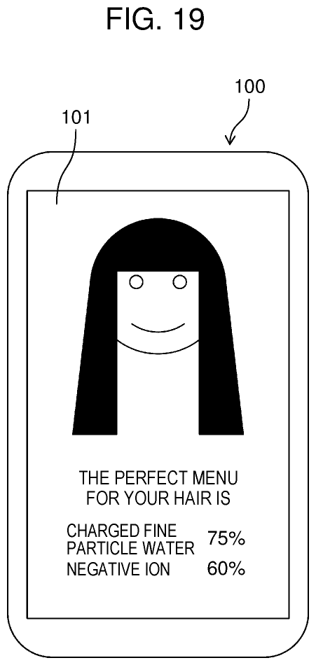

FIG. 19 is a schematic view illustrating a second example of the output screen displayed on terminal display unit 101 (or display 73). The output screen of the second example displays various states in non-real time after hair dryer 1 dries at least a part of the hair or applies a component to at least a part of the hair. As shown in FIG. 19, as a result of using hair dryer 1 by the user, the amount of an effective component for the user's hair may be displayed. In this example, the component amounts of charged fine particle water and negative ions are displayed.

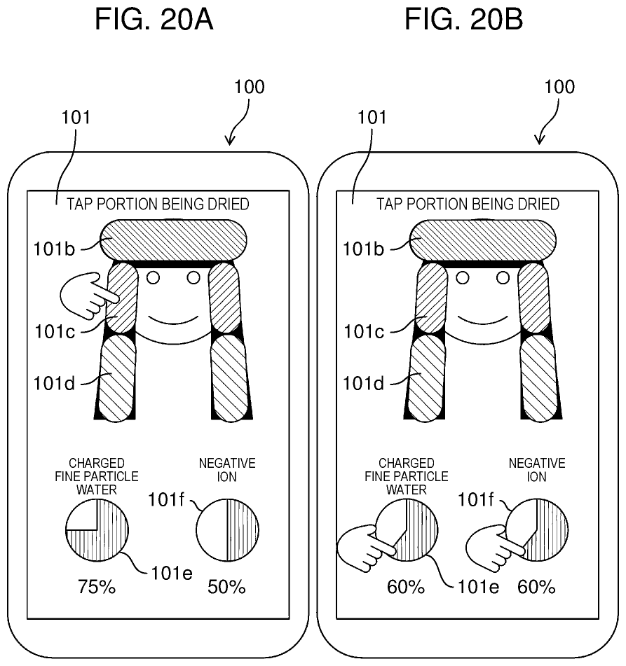

FIGS. 20A and 20B are schematic views illustrating a third example of the output screen displayed on terminal display unit 101 (or display 73). The output screen of the third example displays various states in non-real time, similarly to the output screen of the second example. Further, the output screen of the third example displays the component amount for each part of the user's hair, and displays the part of the hair and the component amount for each part such that the user can change them. First, on the output screen of the third example, a schematic drawing of the user's hair with three types of tap areas is displayed. First tap area 101b corresponds to a part at the root of the hair. Second tap area 101c corresponds to a part at the middle of the hair. Third tap area 101d corresponds to a part at the tip of the hair. Further, on the output screen of the third example, the component amount at the present time is displayed in a pie chart, for example, in addition to numerical values. For example, first pie chart 101e indicates the component amount of charged fine particle water. Second pie chart 101f indicates the component amount of negative ions. FIG. 20A shows, as an example, a state in which the user selects the middle as the part of the hair for which the component amount is desired to be changed by the subsequent operation of hair dryer 1. In this case, the user can select the middle by tapping second tap area 101c on the third output screen.

FIG. 20B shows, as an example, a state in which the user inputs a component amount to be newly set when the user wants to change the component amount by the subsequent operation of hair dryer 1. The user can set a desired component amount by changing the display value while tapping first pie chart 101e and second pie chart 101f on the third output screen.

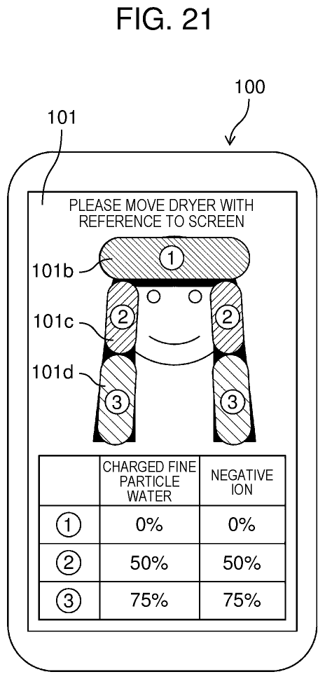

FIG. 21 is a schematic view illustrating a fourth example of the output screen displayed on terminal display unit 101 (or display 73). The output screen of the fourth example displays various states in non-real time, similarly to the output screen of the third example. In the output screen of the third example shown in FIG. 20A, when any one of first tap area 101b, second tap area 101c, and third tap area 101d is tapped, the component amount in the part corresponding to the area is displayed. On the other hand, on the output screen of the fourth example, the component amount for each part of the hair are collectively displayed, and the order of the parts to which the user should apply heat or a component by hair dryer 1 is displayed with numerals. The user can efficiently apply a desired component by moving hair dryer 1 so as to apply heat or a component in the order of the numbers attached to each of first tap area 101b, second tap area 101c, and third tap area 101d.

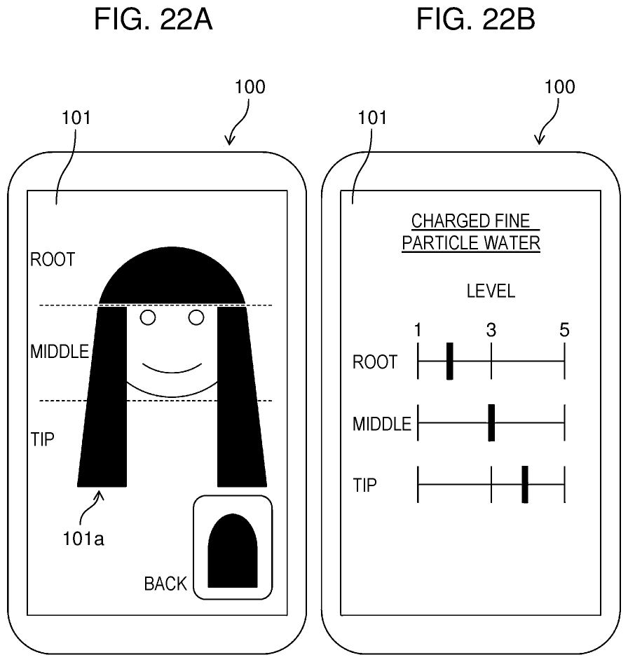

FIGS. 22A and 22B are schematic views illustrating a second example of the input screen displayed on terminal display unit 101 (or display 73). The input screen of the second example corresponds to a case where the user changes the component to be applied to each part of the hair. FIG. 22A shows a first input screen of the second example. Image 101a displayed on the first input screen is a schematic view of the user's front hairstyle, and is a divided image divided into three parts of the root, the middle, and the tip in the vertical direction. On the first input screen, a schematic drawing of the user's rear hairstyle is also displayed. FIG. 22B shows a second input screen of the second example. On the second input screen, a level adjustment screen for the user to change the setting of charged fine particle water is displayed for each area of the divided screen displayed on the first input screen. In a case where the user wants to change charged fine particle water to his or her desired component amount instead of the component amount set by controller 80, first, on the first input screen, three parts in which the amount of the component can be changed in the hair are presented as image 101a. Next, the user can display the second input screen and change the component amount of the charged fine particle water at a plurality of levels so that the component amount of the charged fine water particles becomes a desired component amount for each part.

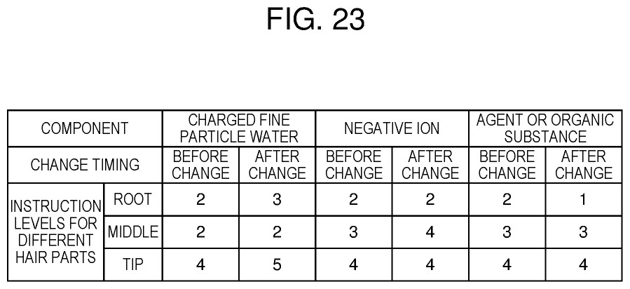

FIG. 23 is a table illustrating a setting example in a case where the component amount is changed for each part of the hair using the input screen of the second example shown in FIG. 22B. In a case where the component to be changed is charged fine particle water as illustrated in FIG. 22B, for example, when the level of the charged fine particle water applied to the root of the hair before the change is “2,” the user may increase the level to “3” by using the input screen of the second example according to his or her preference. The user can change the component amount of the charged fine particle water to a desired component amount by changing the level in the same way in the middle or the tip of the hair, which are other parts of the hair. Further, the user can change the component amount of not only the charged fine particle water but also other components such as negative ions, an agent, and an organic matter, using the input screen of the second example in the same way.

Next, a method for determining an end of drying in a case where the waviness and curl of hair are used as criteria will be described.

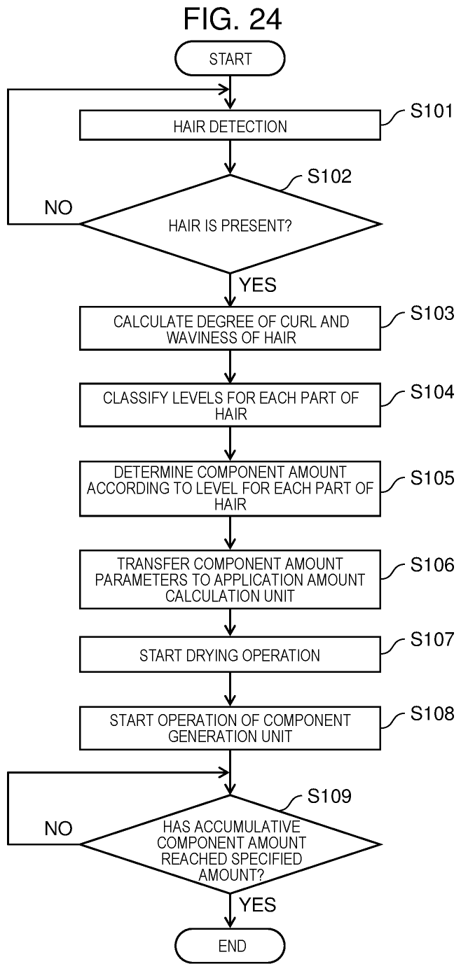

FIG. 24 is a flowchart illustrating a step of determining the end of drying in a case where the waviness and curl of hair are used as criteria. First, controller 80 causes hair detection unit 63 to detect the user's hair (step S101). Next, controller 80 determines whether hair is present based on a result of detection by hair detection unit 63 (step S102). Here, when determining that there is no hair (step S102. NO), controller 80 returns to step S101 and repeats the detection of hair. On the other hand, when determining that the hair is present (step S102: YES), controller 80 then causes, for example, wetting calculation unit 86 to calculate a degree of the waviness and curl of the hair based on the output signal from wetting detection unit 60 (step S103). Next, controller 80 causes, for example, hair characteristic recognition unit 81 to execute a classification of the levels of the waviness and curl for each part of the hair based on the output signal from part detection unit 64 and a calculation result in wetting calculation unit 86 (step S104). Next, controller 80 causes, for example, table generation unit 82 to determine the component amount according to the levels of the waviness and curl for each part of the hair (step S105). Next, controller 80 transfers the parameters related to the component amount determined in step S105 to application amount calculation unit 83 (step S106). Next, controller 80 starts the drying operation, that is, controls heat amount control unit 85 to start applying heat to heat application unit 30 (step S107). Next, controller 80 controls component amount control unit 84 to start applying the component to component generation unit 40 based on component amount parameters transferred to application amount calculation unit 83 (step S108). Next, while component generation unit 40 is operating, controller 80 causes, for example, accumulative calculation unit 88 to calculate the accumulative component amount, and determines whether the accumulative component amount has reached a specified value (step S109). Here, when determining that the accumulative component amount has not reached the specified value (step S109: NO), controller 80 repeats the determination in step S109. On the other hand, when determining that the accumulative component amount has reached the specified value (step S109: YES), controller 80 terminates the operations of heat application unit 30 and component generation unit 40 to terminate the drying.

Next, how controller 80 estimates the degree of dryness of the hair when the user's hair is being dried will be described.

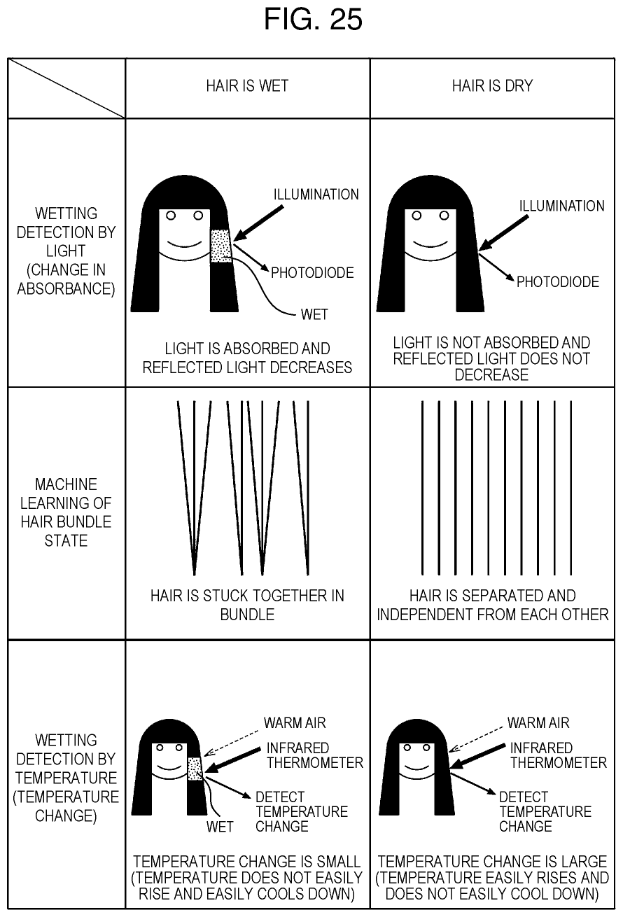

FIG. 25 is a table illustrating several principles that can be used to determine whether hair is wet or dry. To start with, in the present exemplary embodiment, the degree of dryness of the hair is estimated by drying estimation calculation unit 87 based on the wetting information calculated by wetting calculation unit 86. Further, in the present exemplary embodiment, wetting detection unit 60 is specifically wetting detection sensor 60a that is a photodiode. The wetting information is the absorbance calculated by wetting calculation unit 86 based on the signal intensity from wetting detection sensor 60a. As shown in the upper column in FIG. 25, in a case where the hair is wet, a large amount of light is absorbed by the hair when the hair is irradiated with light from illumination unit 72, so that reflected light received by wetting detection sensor 60a is decreased. On the other hand, in a case where the hair is dry, the amount of light absorbed by the hair is small when the hair is irradiated with light from illumination unit 72, so that the reflected light received by wetting detection sensor 60a does not decrease. That is, drying estimation calculation unit 87 can estimate the degree of dryness, in other words, whether the hair is wet or dry, based on the change in absorbance.

Further, as another principle, the degree of dryness of the hair may be calculated by machine learning using a bundle state of the hair as wetting information. In this case, wetting detection unit 60 is a photographing unit such as a camera that photographs hair. Wetting calculation unit 86 is a machine learning calculation unit that discriminates the bundle state of the hair based on a hair image photographed by wetting detection unit 60. As shown in the middle column in FIG. 25, in a case where the hair is wet, the hair is stuck together and bundled. On the other hand, in a case where the hair is dry, the hair is separated and independent from each other. In other words, drying estimation calculation unit 87 can estimate the degree of dryness based on the bundle state of the hair discriminated by machine learning calculation unit.

Furthermore, the degree of dryness of the hair may be calculated by wetting calculation unit 86 using the temperature of the hair as wetting information. In this case, wetting detection unit 60 is a temperature sensor. The temperature sensor may be, for example, an infrared thermometer (an infrared sensor). Wetting calculation unit 86 calculates a temperature as wetting information based on the hair measurement value measured by wetting detection unit 60. As shown in the lower column in FIG. 25, in a case where the hair is wet, when warm air is sent from discharge port 10b toward the hair, the temperature of the surface of the hair is less likely to rise and easily cools down, so that a temperature change is small. On the other hand, in a case where the hair is dry, when warm air is sent from discharge port 10b toward the hair, the temperature of the surface of the hair easily rises and is less likely to cool down, so that the temperature change is large. In other words, drying estimation calculation unit 87 can estimate the degree of dryness based on the change in the temperature of the hair.

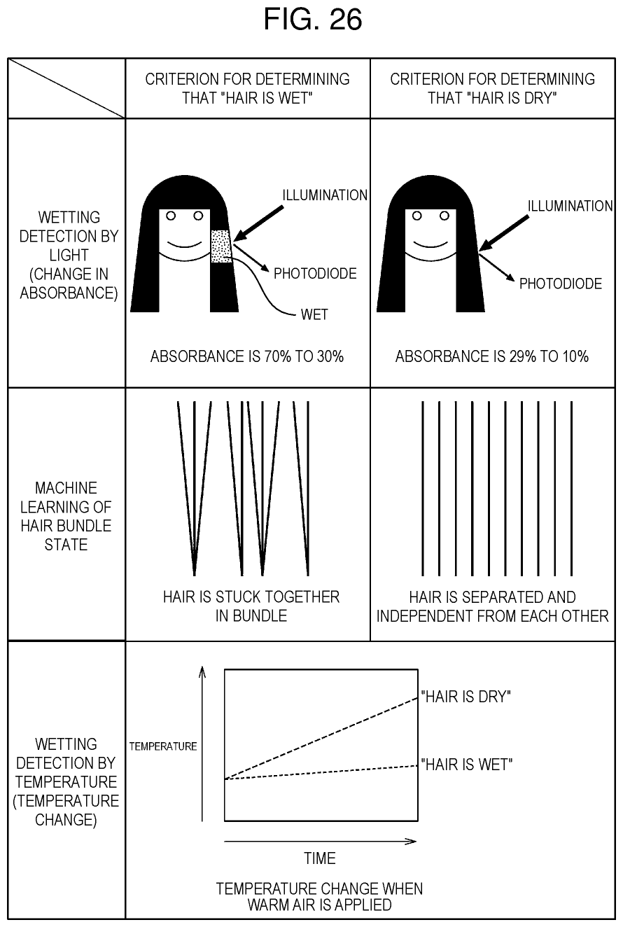

FIG. 26 is a table illustrating specific criteria for determining whether hair is wet or dry, which corresponds to FIG. 25. To start with, when a determination is made based on the change in absorbance as in the present exemplary embodiment, as shown in the upper column in FIG. 26, it may be determined that the hair is wet when the absorbance is 70% to 30%, while it may be determined that the hair is dry when the absorbance is 29% to 10%. Further, when the determination is made based on the bundle state of the hair, the determination may be made according to the result of the machine learning as shown in the middle columns in FIGS. 25 and 26. Furthermore, when the determination is made based on the change in temperature, as shown in the lower column in FIG. 26, it may be determined that the hair is wet when a gradient of the change in temperature when warm air is blown against the hair is gentle, and it may be determined that the hair is dry when the gradient of the change in temperature is steep.

Next, a description will be given of an example of timing related to the degree of dryness of the hair and the component applied by component generation unit 40 or the heat applied by heat application unit 30 with the drying time as a time series.

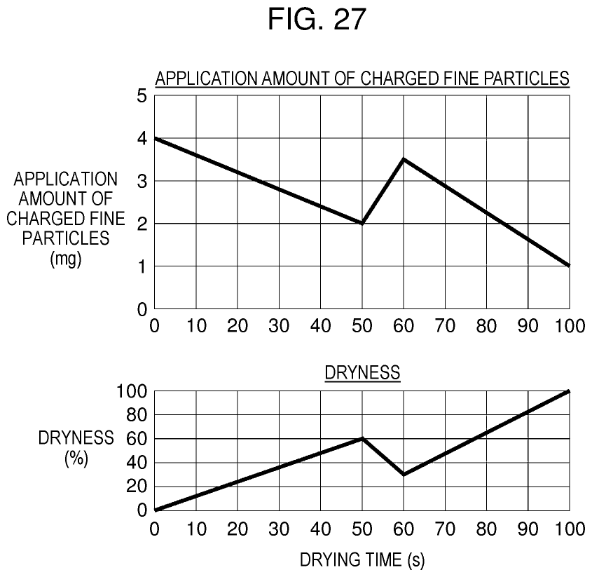

FIG. 27 is a timing chart illustrating an example of a relationship between the application amount of charged fine particles and the degree of dryness of hair. The upper diagram shows the application amount (mg) of the charged fine particles relative to the drying time (seconds). The lower diagram shows the degree of dryness (%) relative to the drying time (seconds). In FIG. 27, the drying times, which are the horizontal axe in the upper diagram and the lower diagram, correspond to each other. Controller 80 may adjust the application amount of charged fine particle water in accordance with the degree of dryness of the hair. Specifically, controller 80 may increase the application amount of the charged fine particle water when the hair is wet, that is, when the degree of dryness is low, and may decrease the application amount of the charged fine particle water when the hair is dry, that is, when the degree of dryness is high.

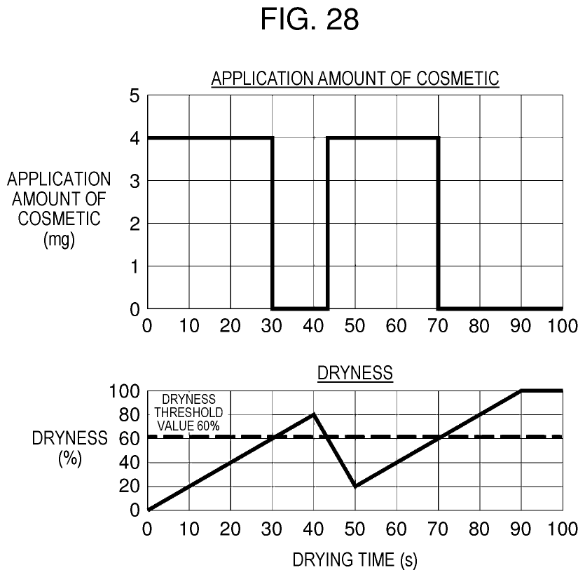

FIG. 28 is a timing chart illustrating an example of a relationship between the application amount of a cosmetic and the degree of dryness of hair. An upper diagram shows the application amount (mg) of the cosmetic relative to the drying time (seconds). The lower diagram shows the degree of dryness (%) relative to the drying time (seconds). In FIG. 28, the drying times, which are the horizontal axe in the upper diagram and the lower diagram, correspond to each other. Controller 80 may adjust the application amount of the cosmetic such that the cosmetic is applied only when the degree of dryness does not exceed a preset threshold value. In the example shown in FIG. 28, controller 80 causes component generation unit 40 to apply, for example, 4 mg of the cosmetic while the degree of dryness does not exceed 60% of the threshold value. In other words, when the degree of dryness exceeds 60% of the threshold value, controller 80 causes component generation unit 40 to stop the application of the cosmetic. In other words, controller 80 may apply the cosmetic only when the hair is relatively wet.

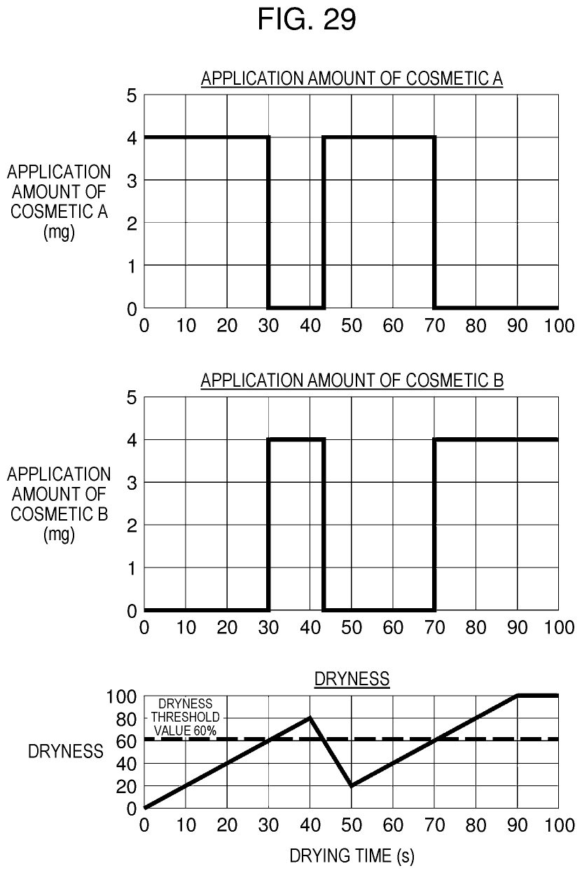

FIG. 29 is a timing chart illustrating an example of a relationship between the application amounts of two kinds of cosmetics A and B and the degree of dryness of hair. The upper diagram shows the application amount (mg) of the cosmetic A relative to the drying time (seconds). The middle diagram shows the application amount (mg) of the cosmetic B relative to the drying time (seconds). The cosmetic A and the cosmetic B are components different from each other. The cosmetic A is an agent desired to be permeated into hair. The cosmetic B is a coating agent. The lower diagram shows the degree of dryness (%) relative to the drying time (seconds). In FIG. 29, the drying times, which are the horizontal axe in the upper diagram, the middle diagram, and the lower diagram, correspond to one another. Controller 80 causes component generation unit 40 to apply, for example, only 4 mg of the cosmetic A only when the degree of dryness does not exceed a preset threshold value, that is, while the hair is relatively wet. On the other hand, controller 80 causes component generation unit 40 to apply, for example, only 4 mg of the cosmetic B only when the degree of dryness exceeds the preset threshold value, that is, when the hair is relatively dry. In other words, controller 80 may particularly apply the cosmetic A that is an agent desired to be permeated while the hair is wet, and may particularly apply the cosmetic B that is a coating agent while the hair is dry.

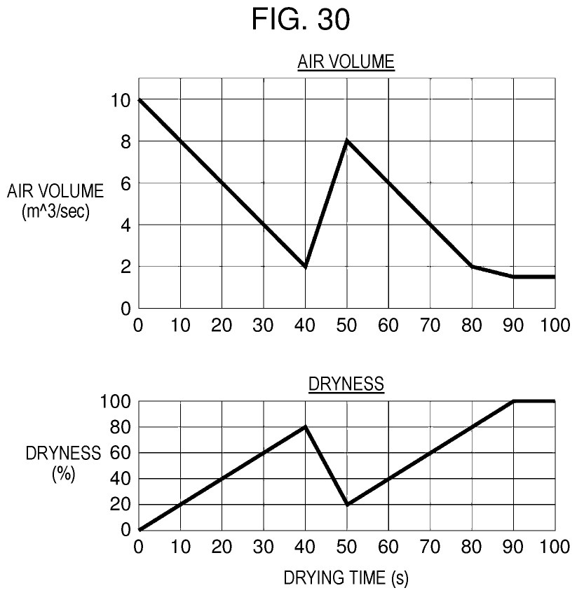

FIG. 30 is a timing chart illustrating an example of a relationship between the air volume and the degree of dryness of hair. The upper diagram shows the air volume (m³/s) relative to the drying time (seconds). The lower diagram shows the degree of dryness relative to the drying time (seconds). In FIG. 30, the drying times, which are the horizontal axe in the upper diagram and the lower diagram, correspond to each other. Controller 80 may adjust the air volume in accordance with the degree of dryness of the hair. Specifically, when the hair becomes dry, controller 80 may decrease the air volume, on the other hand, increase the temperature. As the hair is dried, the glass transition point rises. Therefore, reducing the air volume in association with the drying of the hair allows a curl of the hair to be extended.

Next, effects of hair dryer 1 will be described.

Hair dryer 1 as a hair care device according to the present exemplary embodiment includes heat application unit 30 that applies heat to a user's hair, component generation unit 40 that generates a component acting on the hair, and controller 80 that controls operations of heat application unit 30 and component generator 40. Component generation unit 40 is at least one of an ion generation unit that generates ions, an acidic component generation unit that generates an acidic component, and a charged fine particle water generation unit that generates charged fine particle water. Controller 80 controls the component amount of the component generated by component generation unit 40 based on the hair characteristics of the user.

In the present exemplary embodiment, hair dryer 1 can apply at least one of ions, an acidic component, and charged fine particle water to a user's hair. Further, in hair dryer 1, when heat or a component is applied to the user's hair, controller 80 refers to the hair characteristics of the user, and allows component generation unit 40 to apply the component in a component amount suitable for the hair characteristics of the user. In other words, controller 80 can execute fine control optimal for a user using hair dryer 1.

As described above, the present exemplary embodiment can provide a hair care device that easily provides a finish of hair desired by a user.

Further, in hair dryer 1, the hair characteristics may be waviness or a curl of hair.

Such hair dryer 1 can give many variations to hair characteristics that can be referred to by controller 80, which, as a result, can make it easier to provide a finish of hair desired by the user. In particular, in hair dryer 1 according to the present exemplary embodiment, as the hair characteristics are waviness and a curl of hair, an optimum amount of a component can be applied in accordance with the levels of the waviness and curl. As a result, it is possible to further alleviate the curl, and further improve the touch feeling of hair.

Further, hair dryer 1 includes display 73 that displays at least an image of hair. Controller 80 may change the component amount or type or level of the hair characteristics based on the whole or a part selected by the user in the image on display 73.

In such hair dryer 1, the component amount set by controller 80 or the type or level of the hair characteristics can be changed to the user's preference by using the screen of display 73, so that it can be made easier to provide a finish of hair desired by the user.

Further, hair dryer 1 also includes wetting detection sensor 60a that measures hair. Controller 80 may include drying estimation calculation unit 87 that estimates the degree of dryness of the hair based on the hair measurement value obtained from wetting detection sensor 60a, and application amount calculation unit 83 that adjusts the component amount based on the degree of dryness estimated by drying estimation calculation unit 87.

According to such hair dryer 1, controller 80 adjusts the component amount with reference to the degree of dryness of the hair during the drying operation, so that the component adjusted to a more optimal component amount can be applied to the hair. Hair dryer 1 according to the present exemplary embodiment can particularly suppress a return of the curl caused by wetting of hair because the hair characteristics are waviness and a curl of hair.

Further, hair dryer 1 also includes transmitting and receiving unit 74 that performs transmission and reception with terminal communication unit 103 provided in portable terminal device 100 as an external communication device. Here, it is assumed that at least image 11a of the hair is displayed on terminal display unit 101 provided in portable terminal device 100. At this time, transmitting and receiving unit 74 may receive, from terminal communication unit 103, information related to the whole or part selected by the user in image 11a of terminal display unit 101. Further, controller 80 may change the component amount to a user's desired amount based on the information related to the whole or part of image 101a received by transmitting and receiving unit 74 from terminal communication unit 103.

With such hair dryer 1, the user can adjust the settings in hair dryer 1 from portable terminal device 100, so that convenience for the user can be improved.

### Second Exemplary Embodiment

Hair dryer 1 according to the first exemplary embodiment described above adopts wetting detection sensor 60a (a photodiode) as an example of wetting detection unit 60. On the other hand, in hair dryer 1 according to the second exemplary embodiment, as an example of wetting detection unit 60, any one of the following two photographing units is adopted instead of wetting detection sensor 60a.

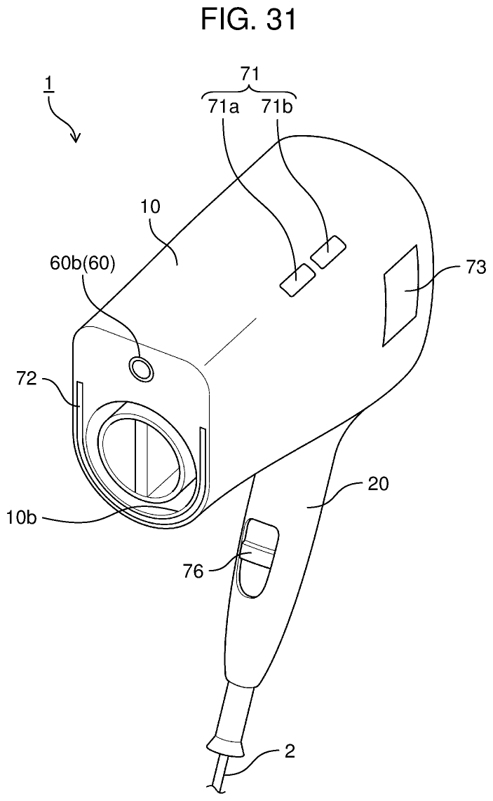

FIG. 31 is a schematic perspective view illustrating a configuration of a first example of hair dryer 1 as a hair care device according to a second exemplary embodiment. Hair dryer 1 according to the first example of the present exemplary embodiment includes photographing unit 60b installed instead of wetting detection sensor 60a in the first exemplary embodiment, and illumination unit 72 installed so as to surround a part of discharge port 10b. Note that, in hair dryer 1 described herein, since the configuration other than photographing unit 60b and illumination unit 72 is the same as the configuration in the first exemplary embodiment (excluding the configuration related to control of, for example, controller 80 and signal processing unit 90), the same reference numerals are given, and a detailed description thereof is omitted.

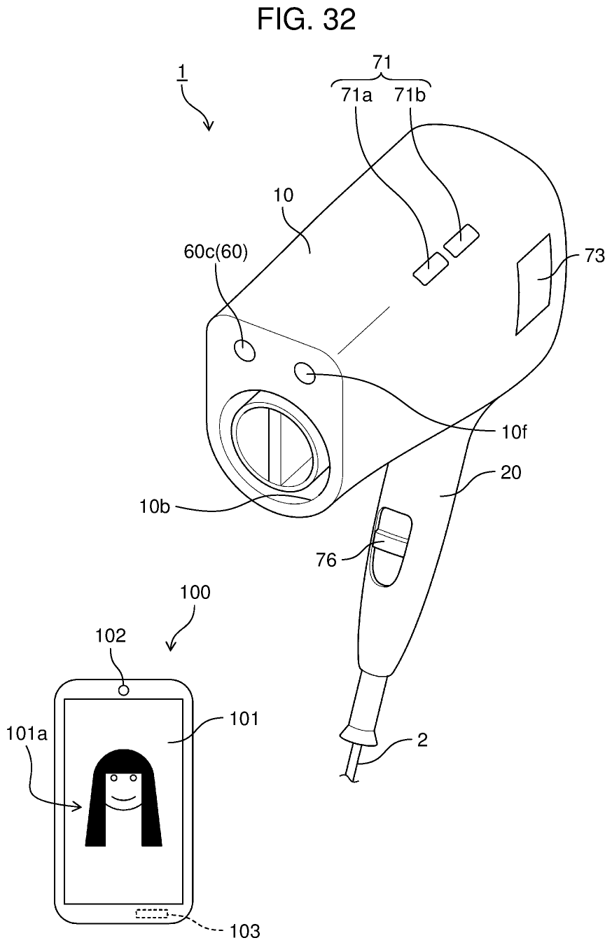

FIG. 32 is a schematic perspective view illustrating a configuration of a second example of hair dryer 1 as a hair care device according to the second exemplary embodiment. Hair dryer 1 according to the second example of the present exemplary embodiment includes transmitting and receiving unit 74, and performs transmission and reception with portable terminal device 100 (terminal communication unit 103) that is an external communication device. Hair dryer 1 described herein uses terminal photographing unit 102 provided in portable terminal device 100 as a photographing unit as wetting detection unit 60. Further, hair dryer 1 according to the second example of the present exemplary embodiment may include temperature sensor 60c (an infrared sensor) installed instead of wetting detection sensor 60a in the first exemplary embodiment. In this case, illumination unit 72 is unnecessary. Note that, in a case where temperature sensor 60c is used as wetting detection unit 60, as described above with reference to FIGS. 25 and 26, drying estimation calculation unit 87 can estimate the degree of dryness based on the temperature change of the hair relative to the drying time. Further, since hair dryer 1 described herein has the same configuration as the configuration in the first exemplary embodiment (excluding the configuration related to control of, for example, controller 80 and signal processing unit 90) except for the configuration described above, the same reference numerals are given, and a detailed description thereof is omitted.

To start with, hair dryer 1 according to the present exemplary embodiment differs from hair dryer 1 according to the first exemplary embodiment in that a photographing unit as described above is adopted. In other words, the first exemplary embodiment and the second exemplary embodiment are similar in that at least one of ions, an acidic component, and charged fine particle water is applied to the user's hair, and in that the component amount is determined with reference to the hair characteristics of the user. Therefore, hair dryer 1 according to the present exemplary embodiment has the same advantageous effects as hair dryer 1 according to the first exemplary embodiment.

Further, in a case where photographing unit 60b or terminal photographing unit 102 is used as wetting detection unit 60, drying estimation calculation unit 87 can estimate the degree of dryness for each drying time by machine learning based on teacher data of a hair image photographed by photographing unit 60b or the like for each drying time. In this case, a point expressed as a “hair measurement value” in the description of hair dryer 1 according to the first exemplary embodiment can be replaced with a “hair image” in the present exemplary embodiment. In other words, even with hair dryer 1 according to the present exemplary embodiment, controller 80 can adjust the component amount with reference to the degree of dryness of the hair during the drying operation, so that the component adjusted to a more optimal component amount can be applied to the hair. Then, hair dryer 1 according to the present exemplary embodiment can suppress a return of the curl caused by wetting of hair because the hair characteristics are waviness and a curl of hair.

Further, in hair dryer 1 according to the present exemplary embodiment, component amount data accumulated based on a hair level determination using the hair image can be used for each drying operation by the user as follows.

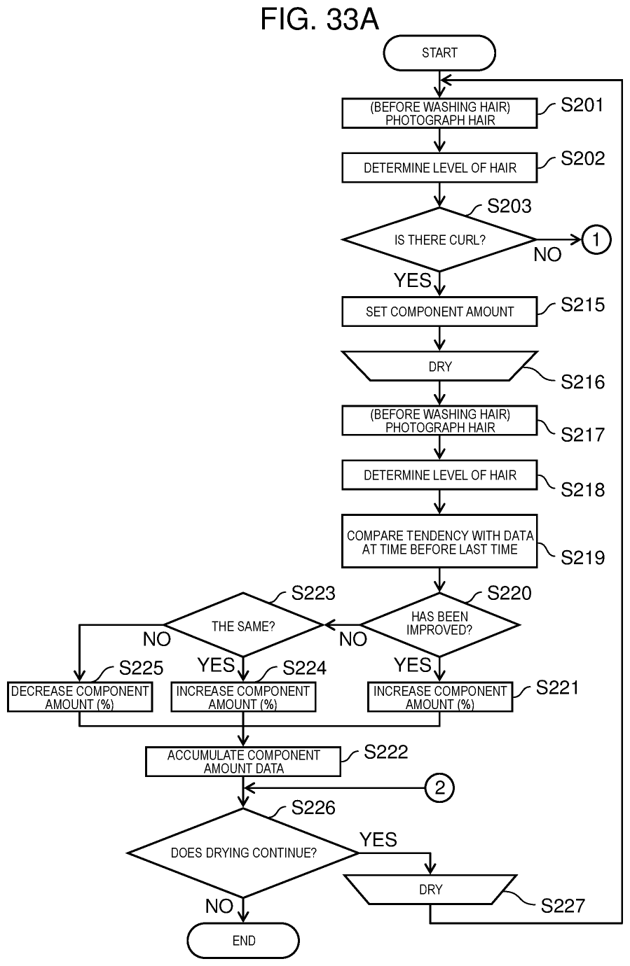

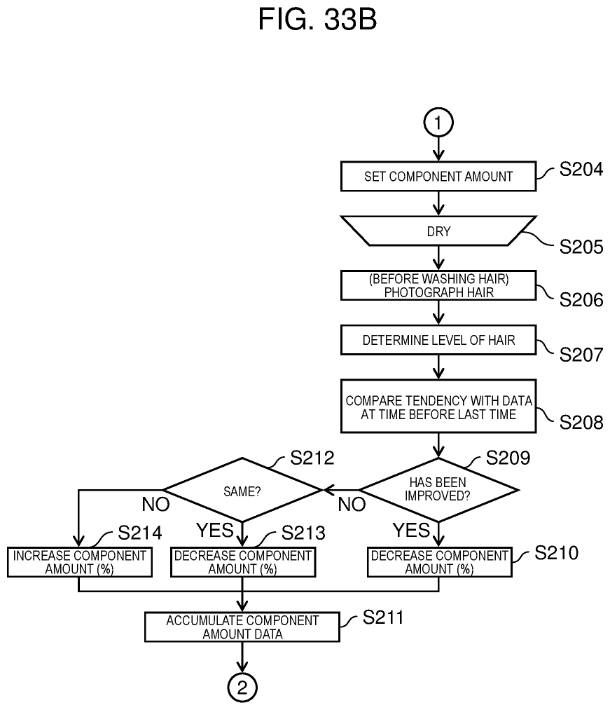

FIGS. 33A and 33B are flowcharts illustrating an example of an operation control step performed by controller 80 in hair dryer 1 according to the present exemplary embodiment.

When the operation control step is started, controller 80 causes photographing unit 60b to photograph the hair, for example, after hair detection unit 63 detects the user's hair, that is, after hair detection unit 63 detects that discharge port 10b of hair dryer 1 is directed to the hair (step S201). Here, the timing of causing photographing unit 60b to photograph the hair is before the user washes the hair or when controller 80 determines that the user's hair is not wet based on the hair measurement value by wetting detection unit 60. Next, controller 80 causes hair characteristic recognition unit 81 to determine the type and level of the hair characteristics based on the hair image obtained in step S201 (step S202). Here, data related to the determined type and level of the hair characteristics is stored in storage 75. Next, controller 80 determines whether there is a curl (or waviness) in the hair with respect to the determination result in step S202 (step S203).

In step S203, when determining that there is no curl in the hair, that is, the hair is straight hair (step S203: NO), controller 80 proceeds to step S204 shown in FIG. 33B.

Referring to FIG. 33B, next, controller 80 causes table generation unit 82 to set the component amount based on the level determination result in step S202 (step S204). Then, when the drying operation is performed by the user, controller 80 causes heat amount control unit 85 to operate heat application unit 30 to dry the user's hair (step S205). At this time, controller 80 causes component amount control unit 84 to operate component generation unit 40 as appropriate to apply the component in the component amount set in step S204. After the drying operation in step S205 is completed, controller 80 is in a standby state until the next drying operation by the user.

Next, when the next drying operation by the user is started, controller 80 causes photographing unit 60b to photograph the hair in the same manner as in step S201 described above (step S206). Next, controller 80 causes hair characteristic recognition unit 81 to determine the type and level of the hair characteristics based on the hair image obtained in step S206 (step S207). Here, data related to the determined type and level of the hair characteristics is stored in storage 75. Next, controller 80 compares a tendency of the data on the hair characteristics determined in step S207 and the data on the hair characteristics determined in step S202 related to the drying operation in the time before the last time, both of which data are stored in storage 75 (step S208).

Next, as a comparison result in step S208, controller 80 determines whether the level of the waviness and curl as the hair characteristics are improved in this time as compared with the time before the last time (step S209). Here, it is assumed that the component to be applied is a component having an optimum amount in accordance with the hair characteristics in order to further improve the hair characteristics. In this case, first, in a case where controller 80 determines that this time is better than the time before the last time (step S209: YES), since the component amount does not need to be increased any more, the component amount is decreased thereafter (step S210). On the other hand, when determining that this time is not better than the time before the last time (step S209: NO), controller 80 proceeds to step S212. Next, in step S212, controller 80 determines whether the level of the waviness and curl as the hair characteristics is the same between the current time and the time before the last time as a comparison result in step S208. Here, when determining that the level of the waviness and curl is the same between the current time and the time before the last time (step S212. YES), controller 80 decreases the component amount that may further improve the hair characteristics (step S213). On the other hand, in a case where controller 80 determines that the level of the waviness and curl is not the same between the current time and the time before the last time, that is, the level of the waviness and curl at the current time is worse than the level of the waviness and curl at the time before the previous time (step S212: NO), the component amount is increased thereafter (step S214). Then, when the component amount is changed in step S210, step S213, or step S214, next, controller 80 stores the changed component amount data in storage 75 (step S211).

On the other hand, in step S203 shown in FIG. 33A, when determining that there is a curl in the hair (step S203: YES), controller 80 proceeds to step S215.

Next, controller 80 causes table generation unit 82 to set the component amount based on the level determination result in step S202 (step S215). Then, when the drying operation is performed by the user, controller 80 causes heat amount control unit 85 to operate heat application unit 30 to dry the user's hair (step S216). At this time, controller 80 causes component amount control unit 84 to operate component generation unit 40 as appropriate to apply the component in the component amount set in step S215. After the drying operation in step S216 is completed, controller 80 is set in a standby state until the next drying operation by the user.

Next, when the next drying operation by the user is started, controller 80 causes photographing unit 60b to photograph the hair in the same manner as in step S201 described above (step S217). Next, controller 80 causes hair characteristic recognition unit 81 to determine the type and level of the hair characteristics based on the hair image obtained in step S217 (step S218). Here, data related to the determined type and level of the hair characteristics is stored in storage 75. Next, controller 80 compares the tendency of the data on the hair characteristics determined in step S218 and the data on the hair characteristics determined in step S202 related to the drying operation in the time before the last time, which are stored in storage 75, respectively (step S219).

Next, as a comparison result in step S219, controller 80 determines whether the level of the waviness and curl as the hair characteristics is improved at the current time as compared with the level of the waviness and curl at the time before the last time (step S220). Here, it is assumed that the component to be applied is a component having an optimum amount in accordance with the hair characteristics in order to further improve the hair characteristics. In this case, first, when determining that the level of the waviness and curl at the current time is better than the level of the waviness and curl at the time before the last time (step S220: YES), controller 80 increases the component amount thereafter (step S221). On the other hand, when determining that the level of the waviness and curl at the current time is not better than the level of the waviness and curl at the time before the last time (step S220: NO), controller 80 proceeds to step S223. Next, in step S223, controller 80 determines whether the level of the waviness and curl as the hair characteristics is the same between the current time and the time before the last time as a comparison result in step S208. Here, in a case where controller 80 determines that the level of the waviness and curl at the current time and the level of the waviness and curl at the time before the last time are the same (step S223: YES), since there is still room for improvement, the component amount is increased thereafter (step S224). On the other hand, in a case where controller 80 determines that the level of the waviness and curl at the current time and the level of the waviness and curl at the time before the previous time are not the same, that is, the level of the waviness and curl at the current time is worse than the level of the waviness and curl at the time before the last time (step S223: NO), since the component amount does not need to be increased any more, the component amount is decreased thereafter (step S225). Then, when the component amount is changed in step S221, step S224, or step S225, controller 80 then stores the changed component amount data in storage 75 (step S222).

Then, after step S211 or step S222, controller 80 determines whether the drying operation is continued (step S226). Here, when determining that the drying operation continues (step S226: YES), controller 80 causes heat amount control unit 85 to operate heat application unit 30 to dry the user's hair (step S227). At this time, controller 80 controls component amount control unit 84 based on the changed component amount data accumulated in step S211 or step S222 to operate component generation unit 40. After the drying operation in step S227 is completed, controller 80 may be set in a standby state until the next drying operation by the user, and thereafter, may return to step S201.

On the other hand, in step S226, when controller 80 determines that the drying operation does not continue (step S226: NO), the operation control step in the present exemplary embodiment ends.

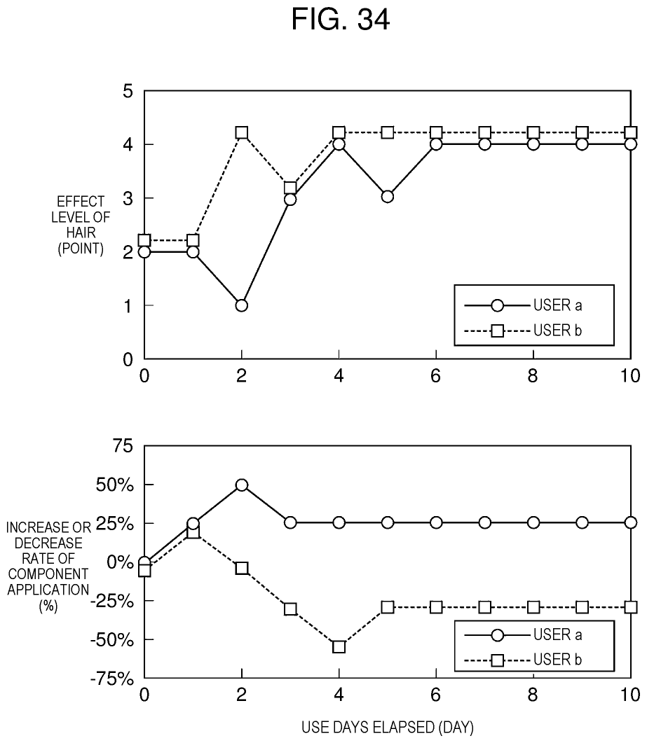

FIG. 34 is a graph illustrating an effect in a case where the operation control step shown in FIGS. 33A and 33B is adopted. The upper diagram shows an effect level (point) of hair relative to use days elapsed (day) of hair dryer 1. The lower diagram shows an increase or decrease rate of the component application (%) relative to the use day elapsed (day) of hair dryer 1. In FIG. 34, the use days elapsed, which are the horizontal axes in the upper diagram and the lower diagram, correspond to each other. Further, FIG. 34 illustrates a case of user a and user b who are two different users.

As described above, first, in the operation control step shown in FIGS. 33A and 33B, the component amount data optimized for each drying operation is accumulated, and in the subsequent drying operation, the component is applied using the most optimized component amount data at that time. Therefore, as shown in each diagram of FIG. 34, for any user, as the number of days on which hair dryer 1 is used increases, the effect on the hair and the increase or decrease rate of the component application become stable.

Further, hair dryer 1 according to the present exemplary embodiment includes photographing unit 60b that photographs hair. Controller 80 may include hair characteristic recognition unit 81 that classifies the hair characteristics based on the hair image obtained by photographing unit 60b, and may cause component generation unit 40 to adjust the component amount for each level of the hair characteristics classified by hair characteristic recognition unit 81.

In such hair dryer 1, controller 80 can automatically classify hair characteristics without the need for the user to directly set the hair characteristics through input unit 71. Further, in hair dryer 1 according to the present exemplary embodiment, hair characteristics are automatically classified as described above, so that, for example, inappropriateness of the component amount due to erroneous recognition by the user can be reduced.

Further, like in the first exemplary embodiment, hair dryer 1 according to the present exemplary embodiment may include wetting detection sensor 60a that measures hair. Controller 80 includes wetting calculation unit 86 that calculates wetting information on wetting of the hair based on the hair measurement value obtained from wetting detection sensor 60a or the hair image obtained from photographing unit 60b. When determining that the user has not washed the hair or the hair is not wet, wetting calculation unit 86 may cause photographing unit 60b to photograph the hair to acquire a hair image, and determine the component amount based on the acquired hair image.

According to such hair dryer 1, controller 80 can acquire the hair characteristics of the user from the hair image when the hair is in the normal condition, and thus can set a more optimal amount of the component to be applied.

Furthermore, hair dryer 1 according to the present exemplary embodiment includes storage 75 that accumulates information related to the hair image obtained from photographing unit 60b. Controller 80 may refer to the information related to at least two or more hair images stored in storage 75 according to an instruction of the user, and cause display 73 to display the change in the level of the hair characteristics.

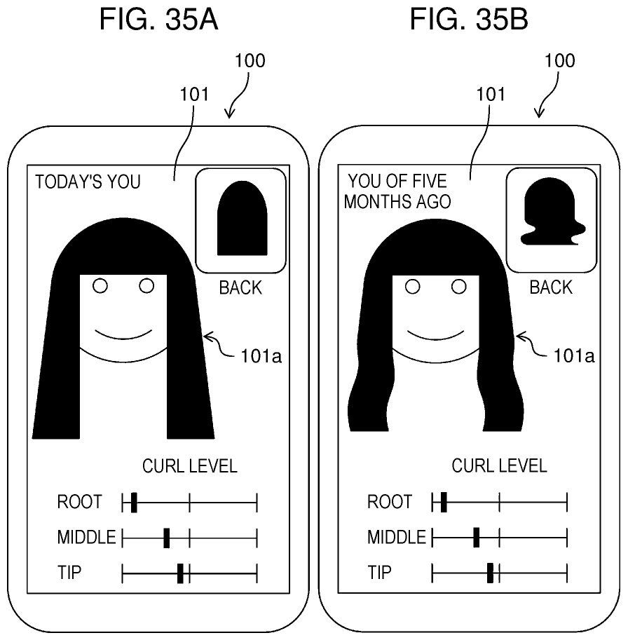

FIGS. 35A and 35B are schematic views illustrating a fifth example of the output screen displayed on terminal display unit 101 (or display 73). The output screen of the fifth example shows a change in the level of the hair characteristics of each user, and as an example, shows a schematic view of the user's front hairstyle and rear hairstyle and the level of the curl for each part of the hair. FIG. 35A shows the condition of the user's hair today. On the other hand, FIG. 35B shows the condition of the user's hair five months ago as an example. With such an output screen displayed, display 73 (or terminal display unit 101) can present the daily change in the condition of the hair to the user.

### Third Exemplary Embodiment

Furthermore, wetting detection unit 60 may be a moisture content sensor that directly measures the moisture content of the hair by coming into contact with the user's hair.

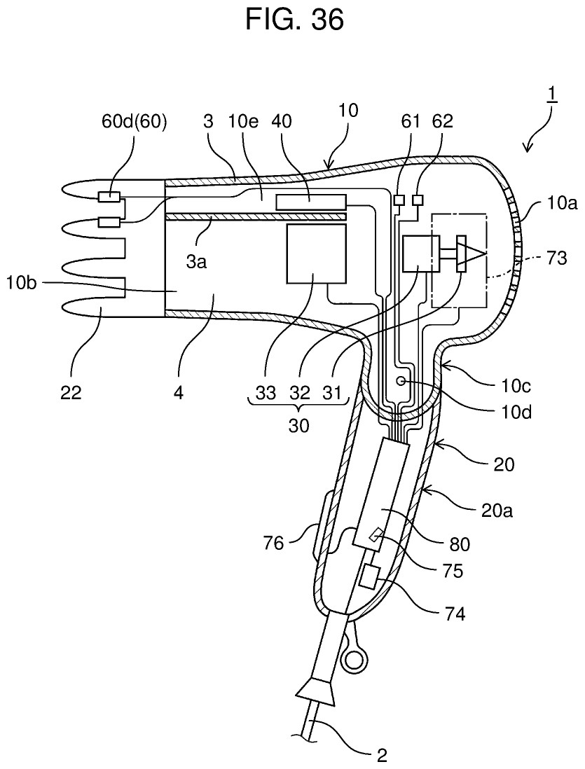

FIG. 36 is a schematic sectional view illustrating a configuration of hair dryer 1 as a hair care device according to a third exemplary embodiment. Hair dryer 1 according to the present exemplary embodiment does not include wetting detection sensor 60a and illumination unit 72 included in hair dryer 1 according to the first exemplary embodiment. On the other hand, hair dryer 1 according to the present exemplary embodiment includes brush part 22 attached to discharge port 10b, and moisture content sensor 60d installed on brush part 22. Here, since the other configurations of hair dryer 1 are the same as those in the first exemplary embodiment (excluding those related to control of controller 80, signal processing unit 90, and the like), the same reference numerals are given, and a detailed description thereof is omitted.

In this case, the hair measurement value is the moisture content of the hair. Moisture content sensor 60d can measure the moisture content of the hair whiile the user dries the hair while applying the brush part 22 to the hair. Then, drying estimation calculation unit 87 can estimate the degree of dryness based on the acquired moisture content.

### Other Exemplary Embodiments

A hair care device according to another exemplary embodiment of the present disclosure may include a hair feature discrimination unit, a substance component amount determination unit, and a substance spraying unit. The hair feature discrimination unit discriminates hair features of the user. The substance component amount determination unit determines the component amount of a substance acting on the user's hair according to the hair features discriminated by the hair feature discrimination unit. The substance spraying unit sprays the substance to the user's hair in the component amount of the substance determined by the substance component amount determination unit. Here, the hair feature discrimination unit may be, for example, an alternative to hair characteristic recognition unit 81 in each of the exemplary embodiments described above. The substance component amount determination unit may be, for example, an alternative to table generation unit 82 in each of the exemplary embodiments described above. Further, the substance spraying unit may be, for example, an alternative to component generation unit 40 in each of the exemplary embodiments described above.

Further, the hair features may be a degree of waviness of hair, a degree of curly hair in hair, a degree of softness and hardness of hair, a degree of bristles in hair, a degree of straight hair in hair, a degree of perm for hair, a degree of thickness of hair, a degree of volume of hair, a degree of gloss of hair, a degree of color of hair, a degree of length of hair, a degree of tension of hair, a degree of elasticity of hair, or a degree of damage to hair.

The substance component amount determination unit may determine the component amount of the substance generated by at least one or more component generation units of the ion component generation unit, the acid component generation unit, and the charged fine particle liquid component generation unit. The ion component generation unit is a component generation unit that generates ions, and may correspond to, for example, second electrostatic atomization device 40b described in the first exemplary embodiment. The acidic component generation unit is a component generation unit that generates an acidic component. The charged fine particle liquid component generation unit is a component generation unit that generates a charged fine particle liquid, and may correspond to, for example, third electrostatic atomization device 40c described in the first exemplary embodiment. Note that, the charged fine particle liquid is a broad expression of the charged fine particle water described above.

The hair feature discrimination unit may include an imaging unit that captures an image of the hairstyle of the user. The imaging unit can be, for example, an alternative to photographing unit 60b in the second exemplary embodiment.

On the other hand, the hair feature discrimination unit may include a living body sensing function unit that senses living body information. Here, the living body information is information on the user's living body, that is, the user's hair, skin, or the like, and refers to, for example, the moisture content and temperature of the hair and skin. The living body sensing function unit can be an alternative to at least one of the various sensors described in the above exemplary embodiments.

Further, the hair feature discrimination unit may include a hair feature input unit to which the user inputs the hair features of the user. The hair feature input unit may be, for example, an alternative to input unit 71 in each of the exemplary embodiments described above or display 73 having an input capability.

The hair feature input unit may cause the user to input the hair features related to the whole or part of the user's hair.

On the other hand, the hair feature input unit may cause the user to perform selection input of at least one or more of the degree of waviness of hair, the degree of curly hair in hair, the degree of softness and hardness of hair, the degree of bristles in hair, the degree of straight hair in hair, the degree of perm for hair, the degree of thickness of hair, the degree of volume of hair, the degree of gloss of hair, the degree of color of hair, the degree of length of hair, the degree of tension of hair, the degree of elasticity of hair, and the degree of damage to hair.

Further, the hair feature discrimination unit may include a hair feature level classification unit that classifies the levels of the hair features of the user using at least one or more of the degrees exemplified above.

Further, the hair feature discrimination unit may discriminate the hair features related to the whole or part of the user's hair.

Alternatively, the hair feature discrimination unit may include a hair feature level classification unit that classifies the levels of the hair features related to the whole or part of the user's hair using at least one or more of the degrees exemplified above.

The hair feature input unit may have a screen for input and output. The screen herein may be, for example, an alternative to display 73 or terminal display unit 101 in each of the exemplary embodiments described above.

Further, the hair care device according to the present exemplary embodiment may include a data transmission and reception unit for performing transmission and reception with outside of the hair care device. The data transmission and reception unit may be, for example, an alternative to transmitting and receiving unit 74 in each of the exemplary embodiments described above.

Furthermore, the hair feature input unit may be provided separately from the hair care device according to the present exemplary embodiment. The hair feature input unit in this case may be, for example, terminal display unit 101 included in portable terminal device 100 in each of the exemplary embodiments described above.

Note that, since the exemplary embodiments described above are intended to illustrate the technique in the present disclosure, various changes, replacements, additions, omissions, and the like may be made within the scope of the claims or equivalents thereof.

### INDUSTRIAL APPLICABILITY

The present disclosure is applicable to all hair care devices for home use or business use that dry a user's hair or adjust a hairstyle of a user.

### REFERENCE MARKS IN THE DRAWINGS

1 hair dryer
2 power supply cord
3 housing
3a partition plate
4 air blowing flow channel
5 main body
10a suction port
10b discharge port
10c connecting part
10d connecting shaft
10e branch channel
10f component discharge port
10g front surface portion
14 nozzle part
20 grip part
20a housing
22 brush part
30 heat application unit
31 fan
32 motor
33 heating unit
40 component generation unit
40a first electrostatic atomization device
40b second electrostatic atomization device
40c third electrostatic atomization device
41a mist atomizer
41b tank
41c pump
41d GND electrode
41e high voltage circuit
41f pump drive circuit
42a discharger
42b GND electrode
42c high voltage circuit
43a discharger
43b Peltier element
43c GND electrode
43d high voltage circuit
50 measurement unit
60 wetting detection unit
60a wetting detection sensor
60b photographing unit
60c temperature sensor
60d moisture content sensor
61 room temperature sensor
62 humidity sensor
63 hair detection unit
64 part detection unit
71 input unit
71a hair waviness input unit
71b hair curl input unit
72 illumination unit
73 display
74 transmitting and receiving unit
75 storage
76 power supply switch
80 controller
81 hair characteristic recognition unit
82 table generation unit
83 application amount calculation unit
84 component amount control unit
85 heat amount control unit
86 wetting calculation unit
87 drying estimation calculation unit
88 accumulative calculation unit
90 signal processing unit
91 part calculation unit
92 initial position determination unit
100 portable terminal device
101 terminal display unit
101a image
101b first tap area
101c second tap area
101d third tap area
101e first pie chart
101f second pie chart
102 terminal photographing unit
103 terminal communication unit

1. A hair care device comprising:
a heat application unit that applies heat to hair of a user;
a component generation unit that generates a component acting on the hair; and
a controller that controls operations of the heat application unit and the component generation unit,
wherein
the component generation unit is at least one of an ion generation unit that generates ions, an acidic component generation unit that generates an acidic component, and a charged fine particle water generation unit that generates charged fine particle water, and
the controller controls a component amount of the component generated by the component generation unit based on hair characteristics of the user.

2. The hair care device according to claim 1, wherein the hair characteristics are waviness or a curl of the hair.

3. The hair care device according to claim 1, comprising a wetting detection sensor that measures the hair,
wherein
the controller includes a drying estimation calculation unit that estimates a degree of dryness of the hair based on a hair measurement value obtained from the wetting detection sensor, and
an application amount calculation unit that adjusts the component amount based on the degree of dryness estimated by the drying estimation calculation unit.

4. The hair care device according to claim 1, comprising a photographing unit that photographs the hair,
wherein the controller includes a hair characteristic recognition unit that classifies the hair characteristics based on a hair image obtained by the photographing unit, and causes the component generation unit to adjust the component amount for each level of the hair characteristics classified by the hair characteristic recognition unit.

5. The hair care device according to claim 4, comprising a wetting detection sensor that measures the hair,
wherein the controller includes a wetting calculation unit that calculates wetting information on wetting of the hair based on the hair measurement value obtained from the wetting detection sensor or the hair image obtained from the photographing unit, and when the wetting calculation unit determines that the user has not washed hair or the hair is not wet, the controller causes the photographing unit to photograph the hair to acquire the hair image, and determines the component amount based on the acquired hair image.

6. The hair care device according to claim 5, wherein
the controller includes a drying estimation calculation unit that estimates a degree of dryness of the hair based on the hair measurement value or the hair image, and
an application amount calculation unit that adjusts the component amount based on the degree of dryness estimated by the drying estimation calculation unit.

7. The hair care device according to claim 4, comprising:
a storage that accumulates information related to the hair image obtained from the photographing unit, and
a display that displays at least an image of the hair,
wherein the controller refers to the information related to at least two or more of the hair images stored in the storage in response to an instruction of the user, and causes the display to display a change in a level of the hair characteristics.

8. The hair care device according to claim 1, comprising a display that displays at least an image of the hair,
wherein the controller changes the component amount or type or level of the hair characteristics based on a whole or a part selected by the user in the image on the display.

9. The hair care device according to claim 1, comprising a transmitting and receiving unit that performs transmission and reception with a terminal communication unit provided in a portable terminal device as an external communication device,
wherein
when at least an image of the hair is displayed on a terminal display unit provided in the portable terminal device,
the transmitting and receiving unit receives, from the terminal communication unit, information related to the whole or part selected by the user in the image on the terminal display unit, and
the controller changes the component amount to a desired amount of the user based on the information related to the whole or part of the image received by the transmitting and receiving unit from the communication unit.

10. A hair care device comprising:
a hair feature discrimination unit that discriminates hair features of a user;
a substance component amount determination unit that determines a component amount of a substance acting on hair of the user according to the hair features discriminated by the hair feature discrimination unit; and
a substance spraying unit that sprays the substance to the hair of the user in the component amount of the substance determined by the substance component amount determination unit.

11. The hair care device according to claim 10, wherein the hair features are a degree of waviness of hair, a degree of curly hair in hair, a degree of softness and hardness of hair, a degree of bristles in hair, a degree of straight hair in hair, a degree of perm for hair, a degree of thickness of hair, a degree of volume of hair, a degree of gloss of hair, a degree of color of hair, a degree of length of hair, a degree of tension of hair, a degree of elasticity of hair, or a degree of damage to hair.

12. The hair care device according to claim 10, wherein the substance component amount determination unit determines the component amount of the substance generated by at least one or more of an ion component generation unit that generates ions, an acid component generation unit that generates an acid component, and a charged fine particle liquid component generation unit that generates a charged fine particle liquid.

13. The hair care device according to claim 10, wherein the hair feature discrimination unit includes an imaging unit that captures an image of a hairstyle of the user.

14. The hair care device according to claim 10, wherein the hair feature discrimination unit includes a living body sensing function unit that senses living body information.

15. The hair care device according to claim 10, wherein the hair feature discrimination unit includes a hair feature input unit to which the user inputs the hair features of the user.

16. The hair care device according to claim 15, wherein the hair feature input unit causes the user to input the hair features related to the whole or part of the hair of the user.

17. The hair care device according to claim 15, wherein the hair feature input unit causes the user to perform selective inputting of at least one or more degrees of the degree of waviness of hair, the degree of curly hair in hair, the degree of softness and hardness of hair, the degree of bristles in hair, the degree of straight hair in hair, the degree of perm for hair, the degree of thickness of hair, the degree of volume of hair, the degree of gloss of hair, the degree of color of hair, the degree of length of hair, the degree of tension of hair, the degree of elasticity of hair, and the degree of damage to hair.

18. The hair care device according to claim 11, wherein the hair feature discrimination unit includes a hair feature level classification unit that classifies levels of the hair features of the user using at least one or more of the degrees.

19. The hair care device according to claim 10, wherein the hair feature discrimination unit discriminates the hair features related to the whole or part of the hair of the user.

20. The hair care device according to claim 11, wherein the hair feature discrimination unit includes a hair feature level classification unit that classifies levels of the hair features related to the whole or part of the hair of the user using at least one or more of the degrees.

21. The hair care device according to claim 15, wherein the hair feature input unit includes a screen for input and output.

22. The hair care device according to claim 10, comprising a data transmission and reception unit that performs transmission and reception with outside of the hair care device.

23. The hair care device according to claim 15, wherein the hair feature input unit is provided separately from the hair care device.
